(19) United States
(12) Patent Application Publication
(10) Pub. No.: US 2014/0369934 A1
(43) Pub. Date: Dec. 18, 2014

(54) dsRNA/DNA Hybrid Genome Replication Intermediate Of Metakaryotic Stem Cells

(71) Applicant: MASSACHUSETTS INSTITUTE OF TECHNOLOGY, Cambridge, MA (US)

(72) Inventors: William G. Thilly, Winchester, MA (US); Elena V. Gostjeva, Winchester, MA (US); B. David Stollar, Newton Highlands, MA (US)

(21) Appl. No.: 14/122,055

(22) PCT Filed: Jun. 1, 2012
(86) PCT No.: PCT/US2012/040361

§ 371 Date: Aug. 21, 2014

Related U.S. Application Data
(60) Provisional application No. 61/492,738, filed on Jun. 2, 2011.

(51) Int. Cl. C12Q 1/68; A61K 49/00

(52) U.S. Cl. CPC C12Q 1/6881; A61K 49/0004; C12Q 2600/136
USPC 424/9.2; 435/6.19

(57) ABSTRACT

The invention provides methods of identifying metakaryotic stem cells, as well as methods of identifying agents that selectively modulate the growth, migration, replication, and/or survival of these cells by detecting an intermediate dsRNA/DNA duplex genome. Also provided are diagnostic, prognostic, and treatment methods for disorders, such as atherosclerosis, restenosis, and benign or malignant tumors.

### RELATED APPLICATIONS

This application claims the benefit of U.S. Provisional Application No. 61/492,738, filed on Jun. 2, 2011. The entire teachings of the above application are incorporated herein by reference.

### BACKGROUND OF THE INVENTION

Scientists have long recognized the resemblance of tumor cells and pathological tissue architectures (such as adenocarcinomas) to the cells and tissues of fetuses of the second trimester (Cohnheim, 1876). Malignant tumors grow at rates similar to early fetuses and like fetal tissues contain niches that are either histologically mesenchymal (locally disorganized) or epithelial (locally organized). Both fetal and cancer stem cells stem cells were expected to increase in number and give rise to the various differentiated cell types populating the highly heterogeneous niches within the organ or tumor mass. Both fetal and cancer stem cells were thus expected to divide symmetrically creating two stem cells and asymmetrically giving rise to a stem cell and a differentiated non-stem cell. Symmetric stem cell divisions would drive the net growth of an organ or tumor while asymmetric divisions would provide the transition cells that themselves divide and provide for the vast majority of cells in the organ or tumor. In a similar vein, non-cancerous hyperproliferative disorders, such as atherosclerosis, or wound healing disorders, such as post-surgical restenosis, are also likely driven by aberrant growth of a stem cell population by symmetric and asymmetric stem cell divisions. It was generally recognized that effective treatments of cancer and other hyperproliferative diseases would require novel therapies directed to selectively modulating the growth, migration, replication, or survival of the stem cells underlying these disorders. Accordingly, there is a need for means to identify the stem cells underlying these pathologies, to identify molecular target molecules and biochemical pathways peculiar to said stem cells and to identify agents that kill them or restrict their growth in patients.

### SUMMARY OF THE INVENTION

The present invention provides, inter alia, methods of identifying stem cells, particularly the metakaryotic stem cells underlying hyperproliferative, wound-healing or tumor disorders—as well as methods of identifying molecular target molecules and biochemical pathways peculiar to said pathological stem cells and to identify agents that kill them or restrict their growth, migration or survival. The invention is based, in part, upon the discovery by applicants that the stem cell lineage that drives human fetal/juvenile growth as well as non-cancerous hyperproliferative, wound healing, and tumor pathologies—called metakaryotes, herein—an unexpected mode of nuclear DNA strand segregation and replication characteristic of metakaryotes but not used by any other known form of plant, animal or bacterial cell type. This novel form of nuclear genomic replication involves formation of a replicative intermediate dsRNA/DNA hybrid (also referred to as a dsRNA/DNA helix duplex, dsRNA/DNA duplex, dsRNA/DNA helix, or simply dsRNA/DNA) genome. This observation is in contrast to previous observations of human eukaryotic non-stem cell division wherein nuclear DNA synthesis proceeds by iterative copying of subchromatid lengths of DNA followed by chromatid condensation and separation at mitosis in double stranded DNA (dsDNA/DNA) form. However, evidence has been proffered that some or all of human cell mitochondrial DNA in non-stem eukaryotic cells are synthesized via dsRNA/DNA intermediates. Specifically, the literature contains no reference to, or suggestion of, dsRNA/DNA hybrid genome in any form of nuclear genomic replication and/or segregation.

Accordingly, in one aspect, the invention provides methods for identifying a metakaryotic stem cell and, in particular, a metakaryotic stem cell undergoing nuclear replication and segregation. The methods include the step of visualizing the nuclei of cells in a sample, e.g., in a tissue or tumor sample or cell culture, where the sample is prepared by a method that substantially preserves the integrity of hollow, bell shaped metakaryotic nuclear structures in nuclei having maximum diameters up to about 50 microns and then identifying metakaryotes by their bell-shaped nuclei and/or recognizing metakaryotic cells undergoing metakaryotic amitosis (i.e., associated with an intermediate dsRNA/DNA duplex genome) in the sample. In certain embodiments, the methods may further include the step of enumerating the cells identified. In certain embodiments, the methods may further include the step of isolating the cells identified. In other embodiments, the methods further comprise identifying macromolecules colocalized with the intermediate dsRNA/DNA duplex genome.

In particular embodiments, the metakaryotic stem cell is a mammalian stem cell, e.g., a human cell. In certain embodiments, the cell may either be a fetal, juvenile, adult, and/or tumor stem cell and/or a stem cell of hyperproliferative pathological lesions as in atherosclerosis, venisclerosis, post-surgical restenosis

Cells identified by the methods of the invention may be in an isolated sample of tissue, tumor biopsy or other hyperproliferative pathological lesions as in atherosclerosis, venisclerosis, post-surgical restenosis or a cell culture sample. In particular embodiments, the cells are cultured and the culture has been irradiated or otherwise treated prior to visualizing the nuclei. In more particular embodiments, the cultured cells are treated to preferentially kill and remove from observation most eukaryotic non-stem cells prior to observation of metakaryotic stem cells. Examples of such treatments include x-irradiation at a dose greater than 400 rads or exposure to chemical agents at concentrations known cause the death of eukaryotic non-stem cells but, to a significantly lower extent the death of metakaryotic cells (e.g., 5-fluorouracil, methotrexate, colchicine).

In other embodiments, the cells are in a tissue biopsy from tissue suspected of containing a tumor, wound healing lesion (e.g., restenotic lesion), atherosclerotic, or venosclerotic lesion.

Nuclei of metakaryotic stem cells undergoing genomic replication and segregation employing a dsRNA/DNA hybrid genome can be visualized by a variety of means and in particular embodiments, visualization can be a result of contacting the cells of a sample with a detectably labeled antibody specific for a dsRNA/DNA duplex. In more particular embodiments, the antibody is fluorescently labeled. In other embodiments, the cells are visualized by indirect immunofluorescence, for example, by contacting them with an antibody specific for and then using a detectably labeled secondary antibody to detect the primary antibody.

In other embodiments, nuclei are visualized as a result of contacting the cells with a dye that discriminates between single-stranded and double-stranded nucleic acids, such as, for example, acridine orange. In more particular embodiments, the cells in a sample are treated to remove RNA, e.g., by RNAse treatment, before visualization of the nucleic acid contents of the nuclei. In other particular embodiments, the visualization of nuclei is a result of contacting the cells with a detectably labeled antibody specific for ssDNA following treatment to remove RNA, such as a fluorescently labeled antibody specific for ssDNA. Similarly cells containing dsRNA/DNA intermediates may be detected by agents, e.g., actinomycin D, that specifically bind to dsRNA/DNA and are conjugated to fluorescent agents.

In another embodiment nuclei containing large amounts (1-24 picograms) of dsRNA/DNA are recognized by the absence or marked diminution of fluorescence of dyes such as DAPI (4′,6-diamidino-2-phenylindole) and or Hoechst 33258 that are brightly fluorescent when they bind to dsDNA but do not cause fluorescence of nuclei containing only dsRNA/DNA. It is here noted that “Hoechst-negative” nuclei have been isolated from mixtures of cells derived from bone marrow or tumors and said isolates have been reported to be “enriched” for hematopietic or tumor stem cells in transplantation experiments. However, no single cell with a Hoechst-negative nucleus has been reported to have stem cell properties such as a hollow bell shaped nucleus nor has the “Hoechst-negative” condition been previously associated with a cell undergoing genome replication and/or segregation, nor has this condition been associated with a cell nucleus containing large amounts of dsRNA/DNA.

In another aspect, the invention provides methods for identifying macromolecules and/or biochemical pathways peculiar to metakaryotic stem cells undergoing genomic replication and segregation. Inhibition of the biological function(s) of such macromolecules or pathways may be expected to inhibit the growth, migration, replication, and/or survival of a metakaryotic stem cell. These methods include the steps of identifying a cell containing an intermediate dsRNA/DNA duplex genome and detecting a candidate macromolecule (directly or indirectly), where co-localization of the candidate macromolecule with the intermediate dsRNA/DNA duplex genome indicates that the macromolecule is associated with metakaryotic stem cell duplication. In certain embodiments, the candidate macromolecule is detected with a detectably labeled antibody. In some embodiments, metakaryotic nuclei undergoing amitotic division now known to contain large amounts of dsRNA/DNA are examined, e.g., under a microscope for the presence of specific macromolecules or biochemical pathways. Visualization of specific macromolecules can be a result of contacting the cells of a sample with a detectably labeled antibody specific for any gene product encoded by the human genome. In more particular embodiments, the antibody is fluorescently labeled. In other embodiments, the cells are visualized by indirect immunofluorescence, for example, by contacting them with an antibody specific for and then using a detectably labeled secondary antibody to detect the primary antibody. As examples of these particular embodiments applicants offer their discoveries of three specific macromolecules each found in large quantities (≧100,000 molecules per nucleus) colocalized in metakaryotic nuclei undergoing genomic replication and segregation but in interphase metakaryotic nuclei or in any eukaryotic nuclei not detected: DNA polymerase beta, DNA polymerase zeta, and RNAse H1. These and other macromolecules had been hypothesized by them to be necessary parts of the biochemical pathway that converts dsRNA/DNA into dsDNA/DNA form after segregation into separate nuclei by amitosis of a metakaryotic stem cell.

In a separate embodiment the presence of specific macromolecules such as enzymes may be detected by visualizing the creation or destruction of colored or fluorescent enzymatic substrates or products in cells containing metakaryotic nuclei in amitosis containing large amounts of dsRNA/DNA.

In a separate embodiment the presence of specific macromolecules such as specific RNA sequences e.g. mRNAs, iRNAs, may be detected by hybridization with labeled probes specific for each desired RNA sequence. Alternately RNA sequences may be detected by methods such as in situ PCR.

Persons skilled in the art of these and similar techniques may readily apply such immunologic or chemical assays by adaptation of methods provided in the scientific literature or devised by routine experimentation.

These methods include the steps of contacting cells comprising metakaryotic stem cells with bell-shaped nuclei undergoing metakaryotic amitosis with a candidate agent and visualizing the nuclei of cells in a sample, where the sample is prepared by a method that substantially preserves the integrity of nuclear structures in nuclei having maximum diameters up to about 50 microns and determining the presence and/or number of bell-shaped nuclei undergoing metakaryotic amitosis in the cells. By comparing the presence and/or number of bell-shaped nuclei undergoing metakaryotic amitosis in the cells contacted with the candidate agent to the presence and/or number of bell-shaped nuclei undergoing metakaryotic amitosis in control cells comprising metakaryotic stem cells but not contacted with the candidate agent, the skilled artisan can identify agents that modulate an amitosis associated with an intermediate dsRNA/DNA duplex genome in metakaryotic stem cells and thereby modulate the growth, migration, replication, or survival of metakaryotic stem cells, e.g., by detecting a change in the number of bell-shaped nuclei undergoing amitosis associated with an intermediate dsRNA/DNA duplex genome in the cells contacted with the candidate agent, relative to the control cells not contacted with the candidate agent. In different applications, an increase or decrease in the growth, migration, replication, or survival of metakaryotic stem cells may be desirable.

In another aspect, the invention provides methods for identifying an agent that inhibits the growth, migration, replication, and/or survival of a metakaryotic stem cell. More specifically it provides a method to discover if an agent interferes with the process of amitosis necessary for increased cell numbers in pathological growths for which metakaryotic cells comprise a stem cell lineage. These methods include the steps of contacting cells comprising metakaryotic stem cells with bell-shaped nuclei undergoing metakaryotic amitosis with a candidate agent and visualizing the nuclei of cells in a sample, where the sample is prepared by a method that substantially preserves the integrity of nuclear structures in nuclei having maximum diameters up to about 50 microns and determining the presence and/or number of bell-shaped nuclei undergoing metakaryotic amitosis in the cells. By comparing the presence and/or number of bell-shaped nuclei undergoing metakaryotic amitosis in the cells contacted with the candidate agent to the presence and/or number of bell-shaped nuclei undergoing metakaryotic amitosis in control cells comprising metakaryotic stem cells but not contacted with the candidate agent, the skilled artisan can identify agents that modulate an amitosis associated with an intermediate dsRNA/DNA duplex genome in metakaryotic stem cells and thereby modulate the growth, migration, replication, or survival of metakaryotic stem cells, e.g., by detecting a change in the number of bell-shaped nuclei undergoing amitosis associated with an intermediate dsRNA/DNA duplex genome in the cells contacted with the candidate agent, relative to the control cells not contacted with the candidate agent. More particularly, the number of metakaryotic nuclei undergoing amitosis employing dsRNA/DNA as a genomic replicative intermediate may be detected and enumerated by any means cited above for detection of nuclei containing large amounts of dsRNA/DNA. For example the number of metakaryotic nuclei containing large amounts of dsRNA/DNA may be detected by immunofluorescent assays for dsRNA/DNA, bright orange fluorescence of nuclei in RNAse-treated preparations or absence or marked diminution of fluorescence in nuclei in preparations treated with dyes such as DAPI or Hoechst 33258. In a more particular embodiment metakaryotic nuclei in the process of formation of a dsRNA/DNA hybrid genome or in the process of converting dsRNA/DNA into dsDNA/DNA form in segregated sister nuclei may be detected by visualizing both dsDNA/DNA and dsRNA/DNA at the same time, e.g., by simultaneously staining fixed tissue or cells with DAPI for dsDNA/DNA and fluorescent antibody specific for dsRNA/DNA given that said fluorescent label attached to or associated with the antibody fluoresces at a wavelength distinguishable from the blue fluorescence of DAPI.

In certain embodiments, the cultured cells are treated to preferentially kill and remove from observation eukaryotic most non-stem cells prior to observation of metakaryotic stem cells. Examples of such treatments include x-irradiation at a dose greater than 400 rads or exposure to chemical agents at concentrations known cause the death of eukaryotic non-stem cells but to a significantly lower extent the death of metakaryotic cells (e.g, 5-fluorouracil, methotrexate, colchicine).

In some embodiments, the cells are mammalian cells. In more particular embodiments, the mammalian cells are contacted with the candidate agent in vivo, and in still more particular embodiments the mammalian cells are obtained from a xenograft solid tumor.

For both in vivo and in vitro screening methods, nuclei may be visualized by any of the methods described above for identification of metakaryotes or any other method disclosed in the application.

The screening methods provided by the invention can identify a variety of agents that modulate the growth, migration, replication, or survival of metakaryotic stem cells. In some embodiments, the candidate agent targets a replication complex comprising a molecule selected from DNA polymerase beta, DNA polymerase zeta, and/or RNAse H1. In more particular embodiments, the candidate agent disrupts the association of DNA polymerase beta, DNA polymerase zeta, or RNAse H1 with the intermediate dsRNA/DNA duplex genome, or disrupts DNA polymerase beta-mediated, and/or DNA polymerase zeta-mediated DNA replication from an intermediate dsRNA/DNA duplex genome and/or RNAse H1-mediated removal of RNA from the intermediate dsRNA/DNA duplex genome. The screening methods provided by the invention will identify agents that modulate the growth, migration, replication, and/or survival of metakaryotes in a variety of ways. In some embodiments, an increase in the number of bell-shaped nuclei undergoing metakaryotic amitosis in the cells contacted with the candidate agent is detected—for example, the agent inhibits completion of replication and replication intermediates accumulate or the agent stimulates an increase in the number of metakaryotic stem cells undergoing replication. In other embodiments, a decrease in the number of bell-shaped nuclei undergoing metakaryotic amitosis in the cells contacted with the candidate agent is detected, e.g., the agent either inhibits initiation of replication or triggers aberrant replication that leads to elimination of replication intermediates. In other embodiments, e.g., wound healing or pathogenic states such as postsurgical restenosis, an agent that modulates the number of migrating metakaryotic stem cells undergoing metakaryotic amitosis is identified—for example an agent increases or decreases the number of migrating metakaryotic stem cells.

In another aspect, the invention provides methods for treating a disorder in a mammalian subject, for example, a human. These methods include contacting a metakaryotic stem cell in the subject with an agent that modulates the growth, migration, replication, or survival of metakaryotic stem cells. In some embodiments, the agent inhibits a replication complex comprising a molecule selected from DNA polymerase beta, DNA polymerase zeta, and/or RNAse H1. Typically, a metakaryotic stem cell comprises a bell-shaped nucleus undergoing metakaryotic amitosis. In more particular embodiments, the agent is a chemical agent that inhibits the association of DNA polymerase beta, DNA polymerase zeta, and/or RNAse H1 with an intermediate dsRNA/DNA duplex genome.

In some embodiments, the agent comprises a dsRNA/DNA duplex binding moiety. In more particular embodiments, the dsRNA/DNA duplex binding moiety is a monoclonal antibody, or fragment thereof, that is specific for a dsRNA/DNA duplex. In other more particular embodiments, the dsRNA/DNA duplex binding moiety is a polyclonal antibody, or fragment thereof, that is specific for a dsRNA/DNA duplex. In particular embodiments, the antibodies or fragments thereof for use in the methods provided by the invention can bind at least one immunogen selected from poly(A)/poly(dT), poly(dC)/poly(I), and φX174 dsRNA/DNA hybrid.

In some embodiments, the agent used in the treatment methods provided by the invention comprises a second moiety. In more particular embodiments, the second moiety degrades or chemically modifies a dsRNA/DNA duplex. In some embodiments, the second moiety is radioactive.

In particular embodiments, the disorder to be treated by the methods provided by the invention comprises a tumor or a lesion, wherein the lesion is associated with a wound healing disorder or non-cancerous hyperproliferative disorder. In more particular embodiments, the lesion is an atherosclerotic or venosclerotic lesion. In other embodiments, the lesion is associated with a wound healing disorder, such as, a restenoic lesion. The disorders to be treated by the methods provided by the invention can include both monoclonal (driven by a single aberrant metakaryotic stem cell) and polyclonal (driven by two or more aberrant metakaryotic stem cells) disorders.

In another aspect, the invention provides methods for diagnosing a tumor, a non-cancerous hyperproliferative disorder, or a wound healing disorder in a mammalian subject in which applicants teach that metakaryotic cells utilizing a dsRNA/DNA replicating intermediate segregated at amitosis. These methods include the steps of contacting cells comprising metakaryotic stem cells with bell-shaped nuclei undergoing metakaryotic amitosis with a candidate agent and visualizing the nuclei of cells in a sample, where the sample is prepared by a method that substantially preserves the integrity of nuclear structures in nuclei having maximum diameters up to about 50 microns and determining the presence and/or number of bell-shaped nuclei undergoing metakaryotic amitosis in the cells. In a surgical sample or biopsy the skilled artisan can identify the nuclei containing an intermediate dsRNA/DNA duplex genome. More particularly, the number of metakaryotic nuclei undergoing amitosis employing dsRNA/DNA as a genomic replicative intermediate may be detected and enumerated. For example the number of metakaryotic nuclei containing large amounts of dsRNA/DNA may be detected by immunofluorescent assays for dsRNA/DNA, bright orange fluorescence of acridine orange-treated nuclei in RNAse-treated preparations or absence or marked diminution of fluorescence in nuclei in preparations treated with dyes such as DAPI or Hoechst 33258. In a more particular embodiment metakaryotic nuclei in the process of formation of a dsRNA/DNA hybrid genome or in the process of converting dsRNA/DNA into dsDNA/DNA form in segregated sister nuclei may be detected by visualizing both dsDNA/DNA and dsRNA/DNA at the same time, e.g. by simultaneously staining fixed tissue or cells with DAPI for dsDNA/DNA and fluorescent antibody specific for dsRNA/DNA given that said fluorescent label attached to or associated with the antibody fluoresces at a wavelength distinguishable from the blue fluorescence of DAPI. The presence of said nuclei with large amounts of dsRNA/DNA is diagnostic of a cancerous or precancerous lesion or a pathological hyperproliferative disorder such as atherosclerosis or venosclerosis or a wound-healing disorder such as post-surgical restenosis.

In more particular embodiments, the determining step of these methods includes determining the number and/or distribution of metakaryotic stem cells undergoing metakaryotic amitosis utilizing dsRNA/DNA genomic replicative intermediates in the sample. In certain embodiments, the methods may further include the step of prognosing the subject (e.g., a human), where the number and/or distribution of cells undergoing metakaryotic amitosis in the sample indicates a low, medium, or high risk prognosis of a tumor, non-cancerous hyperproliferative disorder, or wound healing disorder, e.g., a surgical biopsy of a prostate gland suspected to become early state of malignancy as opposed to a state of desultory hyper proliferation. In more particular embodiments, the methods may further include the step of administering a suitable prophylaxis to the subject. For example, in the presence of a tumor in a subject, the subject may undergo surgery as well as adjuvant therapy, such as chemotherapy. In particular embodiments, the subject may be administered an agent that modulates the growth, migration, replication, or survival of a metakaryotic stem cell as disclosed herein and/or treated by any of the therapeutic methods disclosed herein. More particularly the agent administered may be an inhibitor of the formation and segregation of the dsRNA/DNA hybrid genome or an inhibitor of the processes that convert the dsRNA/DNA inhibitor into the interphase dsDNA/DNA genomic form. Still more particularly the agent may inhibit the functions of any of the enzymes discovered to be physically associated with the dsRNA/DNA hybrid genome such as those discovered by applicants, DNA polymerase beta, DNA polymerase zeta or RNAse H1.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a micrograph showing a symmetrical amitosis of a human fetal colon metakaryotic stem cell (5-7 weeks). This illustrates one common mode, “stacked cup” of symmetric amitoses used in organogenesis, carcinogenesis and pathogenic vascular lesions in humans. Gostjeva et al., 2006, 2009.

FIG. 2 is a micrograph showing an asymmetrical amitosis of a fetal colonic metakaryotic stem cell. (5-7 wks). Asymmetrical division is an essential quality of a stem cell. Gostjeva et al., 2006, 2009.

FIG. 3. shows quantitative Feulgen cytometry of DNA amount (top panels, purple color; quantitated in bottom graph) during and after metakaryotic stem cell symmetrical amitoses in fetal organs (5-9 wks). Data demonstrated that DNA is doubled (increases from 1× to 2× as shown in y-axis) during and soon after metakaryotic amitoses but the biochemical nature of intermediate form of genome was not revealed. Gostjeva et al., 2009. Note that two rings of condensed DNA at bell mouths is the first to demonstrate DNA doubling. These images established that genomic DNA doubling and segregation were occurring simultaneously during amitosis and one explanation, inter alia, involved segregation of the opposite strands of DNA helices into sister nuclei followed by copying to recreate a dsDNA/DNA genome.

FIG. 4. shows micrographs of metakaryotic replication. Left panels (A): Two metakaryotic amitoses another form of metakaryotic amitosis, “kissing bell,” common in early fetal life using quantitative Feulgen staining with transmitted light (purple for DNA). Right two panels (B & C): Two metakaryotic amitoses in “kissing bell” form pretreated with RNAse to remove ribosomal RNA, which would interfere with the detection of ssDNA, then labeled with acridine orange. Green fluorescence indicates double stranded DNA while orange fluorescence denotes single stranded DNA in such RNAse treated samples. It was later understood that double stranded RNA/DNA was transformed into single stranded DNA in this case by pretreatment with RNAse. Arrow represents inventors' interpretation that formation of double stranded RNA/DNA intermediate begins in the two rings of condensed DNA at bell shaped nucleus mouth and then continues to “spread” to the ˜90% of the genome in the body of the nucleus prior to separation of two sister nuclei.

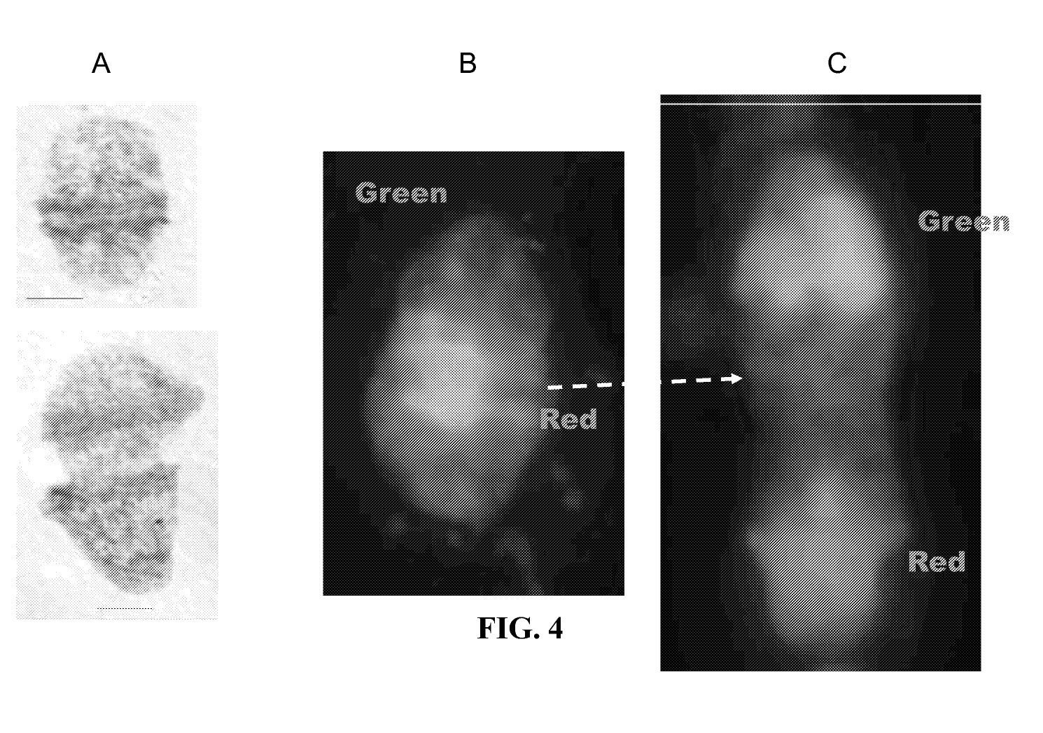

FIG. 5 shows fluorescent micrographs of two metakaryotic multinuclear syncytia of early human fetal development. Specimen was pretreated with RNAse then stained with acridine orange. Top panel (A): syncytium in which no nuclei were labeled orange by treatment. This is an example of a syncytium not undergoing genomic replication at the moment of sampling and RNAse treatment. Bottom panel (B): Syncytium in which all nuclei are labeled orange at the bell mouths indicating the presence of a large amount of dsRNA/DNA in this portion of the nuclei at the time of sampling and staining. This micrograph further indicates that nuclei within the same syncytium undergo synchronous amitosis with genomic doubling via a dsRNA/DNA hybrid genome. Synchronous amitoses in syncytia containing bell shaped metakaryotic nuclei were previously observed with. Feulgen cytometry (Gostjeva et al., 2006).

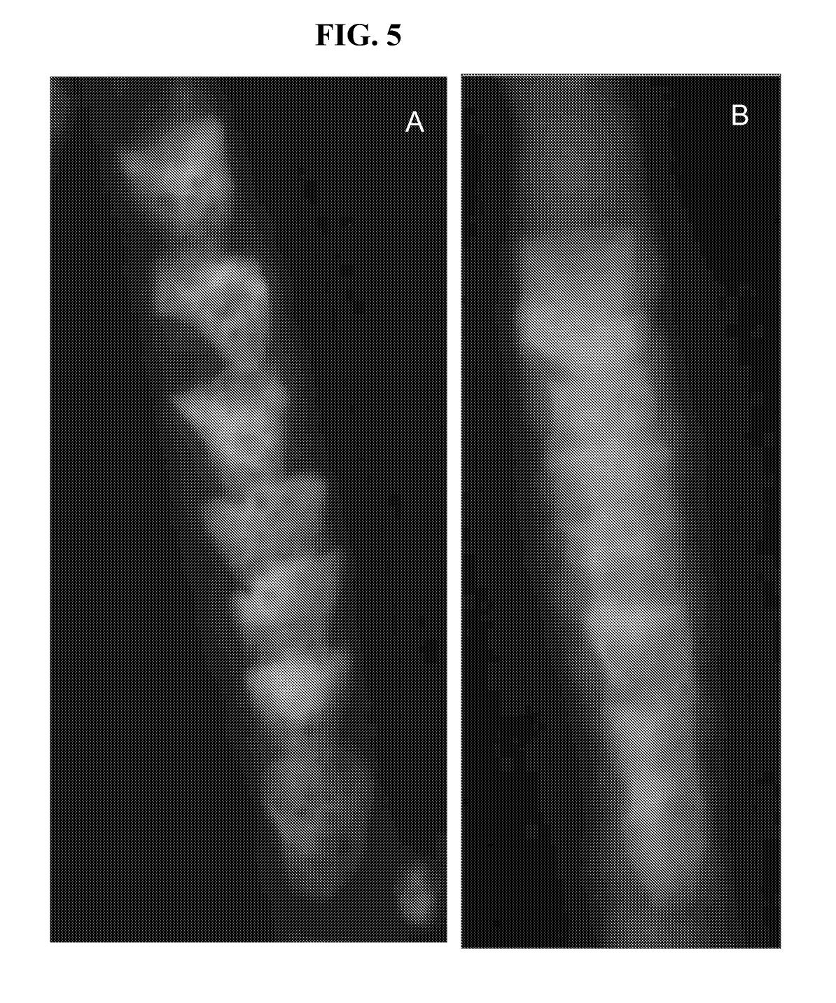

FIG. 6 shows two fluorescent micrographs of multinuclear syncytia of early fetal development using the same tissues, examined in FIG. 5. Antibodies to single stranded DNA (ssDNA) show ssDNA (green fluorescence) in mononuclear and syncytial metakaryotic cells after RNAse pretreatment. The inventors have superimposed a bell shaped template in (A) and arrows in (B) to indicate the bell shape that denotes a metakaryotic nucleus. No signal was observed in the identical specimens not treated with RNAse. Blue fluorescence arises from the dye DAPI that binds to double stranded DNA. This represents an independent means of demonstration that after RNAse treatment amitotic figures of metakaryotic nuclei contained large amounts of a single stranded DNA component.

FIG. 7 shows two fluorescent micrographs. Syncytia were pretreated with RNAse from the same human fetal specimens used in FIGS. 5 and 6 then labeled with green fluorescent antibody to single stranded DNA (A) or with acridine orange (B) which fluoresces orange when bound to single stranded DNA. This figure demonstrates essential identity of label distribution of metakaryotic amitoses after RNAse treatment and staining with independent probes for single stranded DNA. Use of the two physically independent means to recognize ssDNA in RNAse-treated metakaryotic nuclei undergoing amitosis tested and supported the hypothesis that RNA was somehow involved in the metakaryotic genomic intermediate created during amitosis.

FIG. 8. shows the distribution of dsRNA/DNA duplexes within metakaryotic bell shaped nuclei within a tubular syncytium in fetal tissue. FIG. 8A shows that five syncytial bell shaped nuclei contain dsDNA/DNA (DAPI, blue) and dsRNA/DNA (TRITC, red), labeled antibody complex specific for dsRNA/NA duplexes. These five bell shaped nuclei were aligned within syncytium showing association with dividing “bells” within the syncytium. FIG. 8B shows the red fluorescence, which indicated the presence of a dsRNA/DNA duplex binding (TRITC, red), shown without the blue fluorescence from DAPI staining of dsDNA/DNA in the same five bell shaped nuclei. FIG. 8C shows an achromatic image of same five syncytial nuclei showing that they were bell-shaped nuclei as opposed to several other nuclei that were not bell shaped. Scale bar—5 um. Human fetus (9 wks), spinal cord, syncytia Immunofluorescent staining for dsRNA/DNA duplex (AB n3 and TRITC—red). Counterstaining was with DAPI (nucleus—blue).

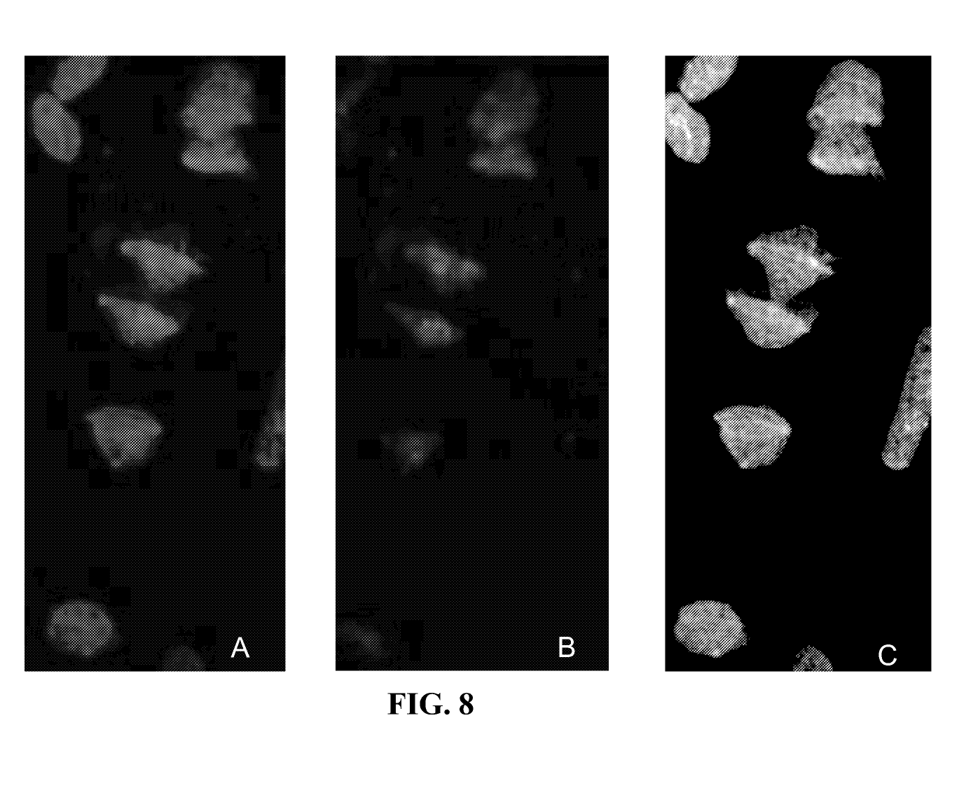

FIG. 9 shows a second set of images illustrating the distribution of dsRNA/DNA duplex within amitotically dividing metakaryotic bell shaped nuclei in fetal tissue. FIG. 9A shows that all four syncytial bell shaped nuclei contained dsDNA/DNA (DAPI, blue) and dsRNA/DNA (TRITC, red), labeled antibody complex specific for dsRNA/DNA duplexes. These four bell shaped nuclei were aligned within the syncytium showing association with dividing “bells” within syncytium. FIG. 9B shows the red fluorescence indicated the presence of a dsRNA/DNA duplex binding (TRITC, red), shown without the blue fluorescence from DAPI staining of dsDNA/DNA in the same four bell shaped nuclei. FIG. 9C shows an achromatic image of same four syncytial nuclei showing that they were bell-shaped nuclei as opposed to another nucleus (upper left) that was not bell shaped. Scale bar—5 um. Human fetus (9 wks), spinal cord, syncytia. Immunofluorescent staining for dsRNA/DNA duplex (AB n3 and TRITC—red). Counterstaining with DAPI (nucleus—blue).

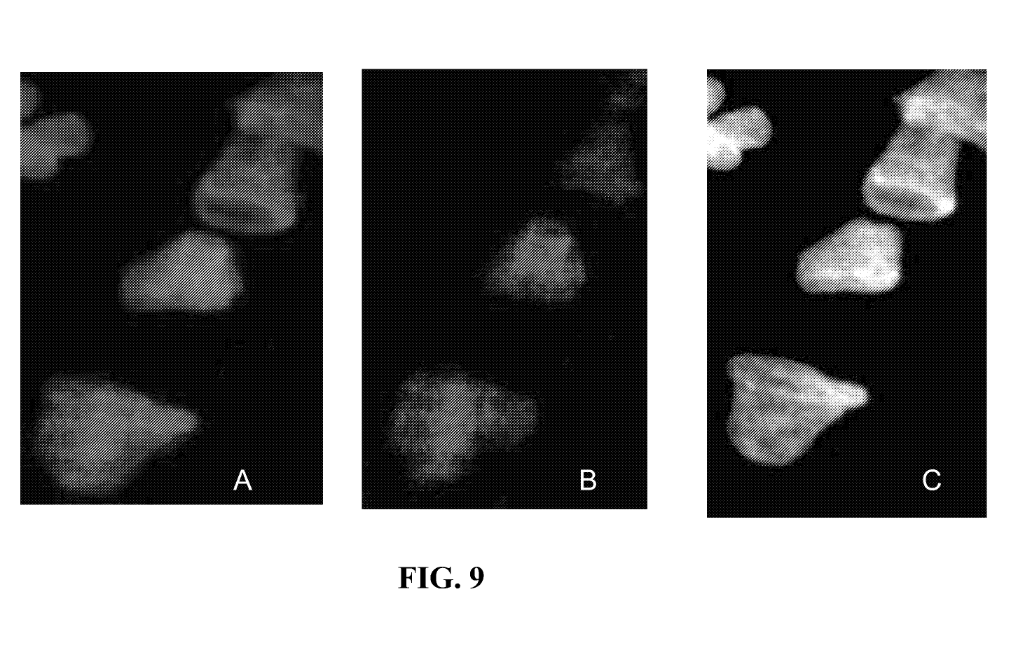

FIG. 10. shows images of bell shaped and derived spherical nuclei during asymmetrical amitosis in a mononuclear metakaryotic cell found among the cells of the HT-29 human colonic adenocarcinoma derived cell line. FIG. 10A shows DAPI-positive blue bell-shaped nucleus (left) and TRITC labeled antibody complex, red spherical eukaryotic nucleus (right). This image teaches that in some instances one nucleus in asymmetrical amitosis may be converted to the interphase dsDNA/DNA form while the other remains at least temporarily in the dsRNA/DNA form of the replicative intermediate. Image also teaches that essentially 100% of the genome of a sister cell produced via amitosis may be comprised of dsRNA/DNA. FIG. 10B shows an achromatic image showing one bell shaped nucleus that does not detect the presence of the dsRNA/DNA mass (arrows). Scale bar—5 um. However, HT-29 cells (human colon adenocarcinoma cell line) Immunofluorescent staining for dsRNA/DNA duplex (AB n2 and TRITC—red). Counterstaining with DAPI (nucleus—blue).

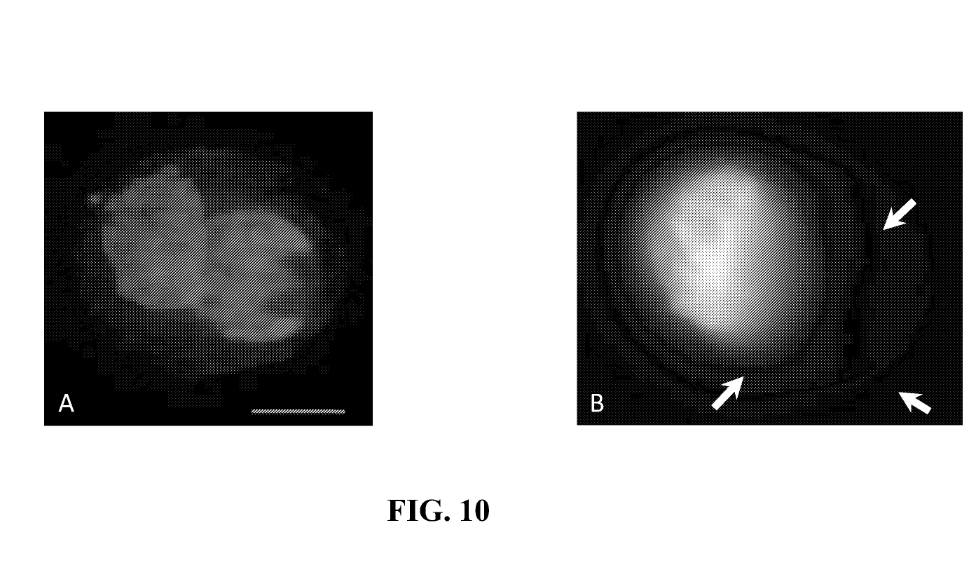

FIG. 11. shows an image of a living, unstained colony of human colonic adenocarcinoma-derived cell line HT-29. Purple, bell shaped object was the nucleus of a metakaryotic cancer stem cell that had just given rise to a eukaryotic cell nucleus seen as the oval body in the mouth of the bell. This image teaches that metakaryotic cell nuclei in the process of amitosis were observed without fixation or dyes using ordinary microscopic or phase contrast optics.

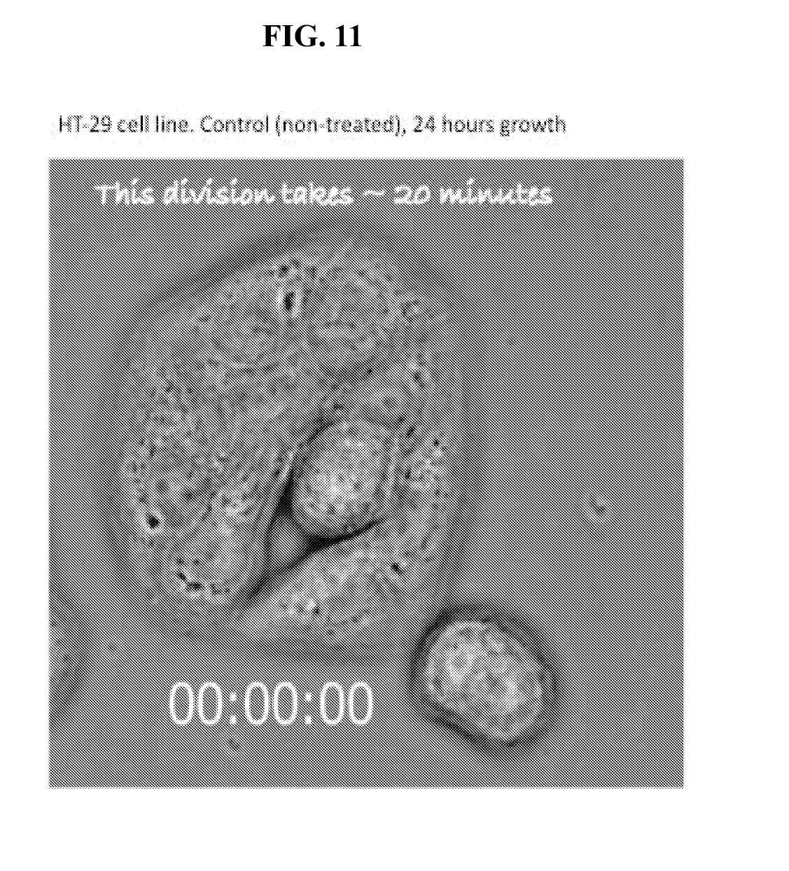

FIG. 12. shows that the purple, bell shaped nucleus of the metakaryotic cells of cell line HT-29, as shown in FIG. 11, was specifically unstained in the presence of Hoechst dyes such as Hoechst 33342 or 33258. All nuclei of eukaryotic cells within the colonies pictured in part were rendered bright blue by the binding of the dye to the dsDNA of the eukaryotic genome including (not shown) eukaryotic cells undergoing mitosis. In the left panel of the upper and lower rows a purple, bell shaped nucleus (arrows) has just given rise to a near-spherical eukaryotic nucleus that subsequently underwent mitosis. In the middle panel of both rows the Hoechst 33342 dye was seen to label all nuclei blue except for the bell shaped metakaryotic stem cell nucleus that emitted no blue fluorescent light and was dubbed a “black hole” by the Applicants when the phenomenon was first observed by them. The right panel of both rows is a composite of the left and middle panels demonstrating that the two images identify the same object, the nucleus of a bell shaped metakaryotic nucleus undergoing mitosis. Applicants note that the dsRNA/DNA hybrid genome discovered by them in metakaryotic amitoses would be predominantly of the “A” form of nucleic acid helix and would not be expected to be rendered fluorescent by dyes such as the Hoechst dyes or DAPI or others that specifically cause “B” form of nucleic acid helices such as dsDNA to fluoresce.

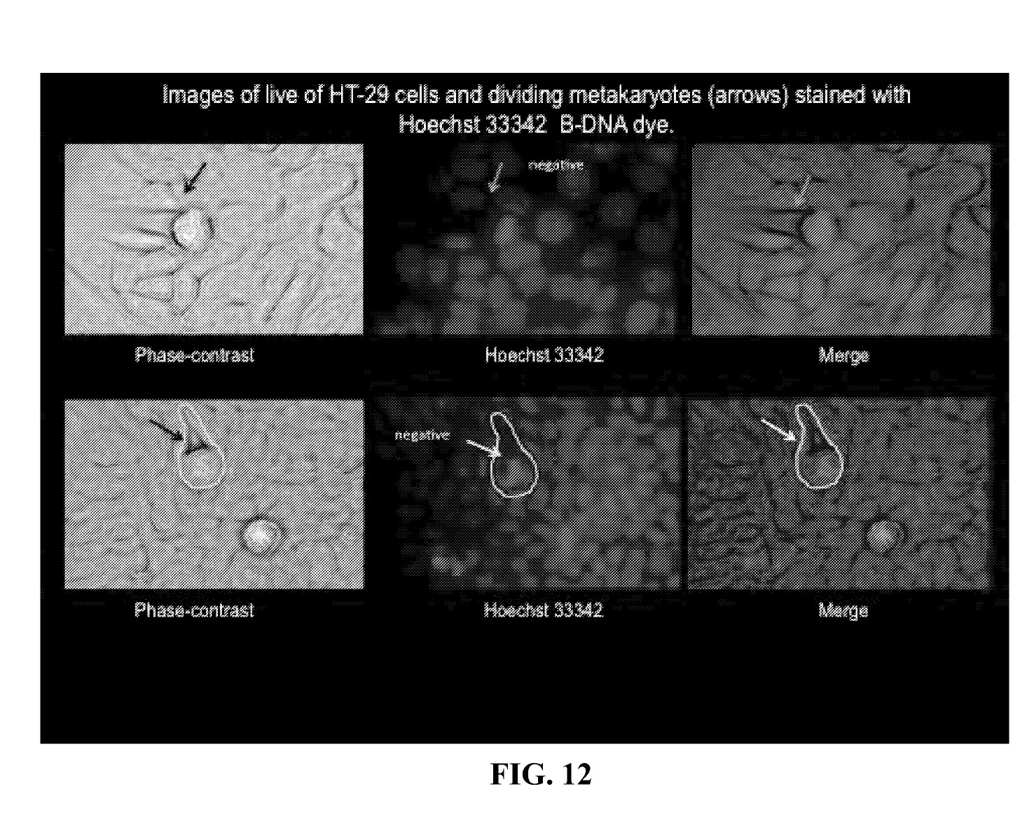

FIG. 13. shows images demonstrating that macromolecules, here enzymes, that are associated with amitosis and genome replication were identified by observing them by their antigenicity in human metakaryotic fetal cells undergoing amitotic divisions that utilize dsRNA/DNA genomic replicative intermediates. Three enzymes are so identified as examples: FIGS. 13 a, d, g: DNA polymerase beta stained by a specific fluorescent (FITC-green) human POL Beta antibody complex. FIGS. 13 b, e, h: DNA polymerase zeta stained by a specific fluorescent (TRITC-red) human POL zeta antibody complex. FIGS. 13 c, f, is RNAse H1 stained by a specific fluorescent (FITC-green) human antibody RNAse H1 antibody complex. FIGS. 13 a, b, c, d, e, f, g, h, i human fetus, spinal cord ganglia, 9 wks. FIGS. 13 a, b, c demonstrate metakaryotic tubular syncytia with dividing bell-shaped nuclei. FIGS. 13 d, e, f demonstrate the symmetrical amitoses in the “kissing-bell” form. FIGS. 13 g, h, i demonstrate various form of symmetric and asymmetric amitoses of metakaryotic bell shaped nuclei.

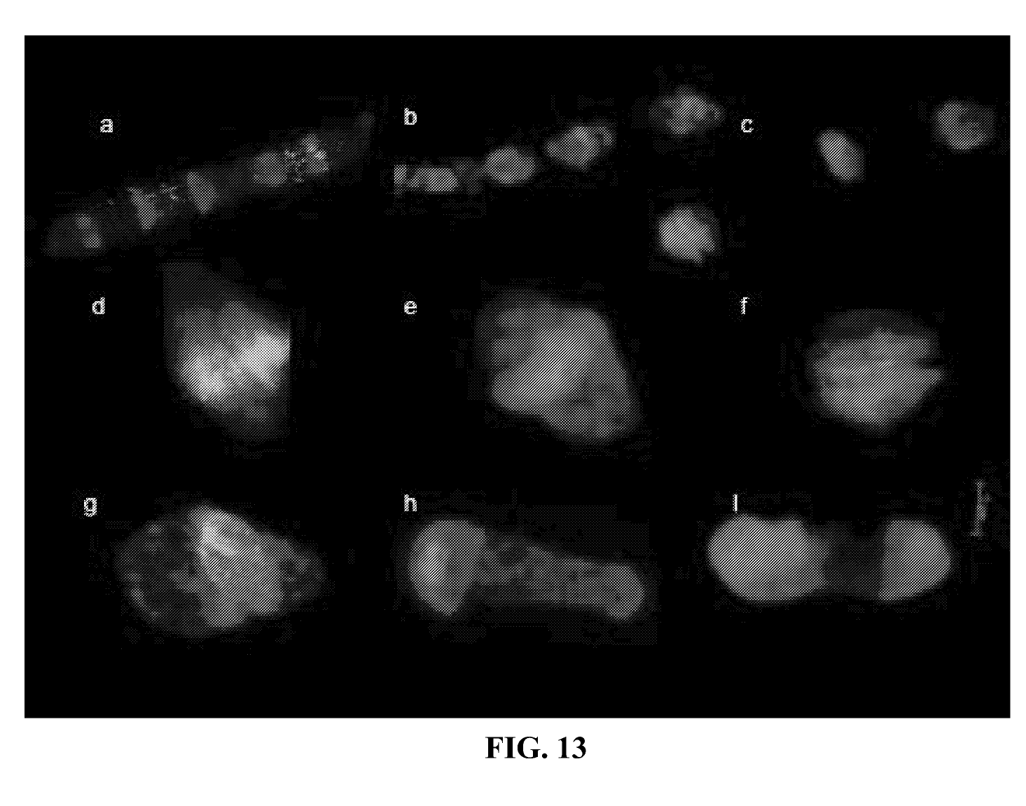

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

### DETAILED DESCRIPTION OF THE INVENTION

A description of example embodiments of the invention follows.

Scientists have long suspected that stem cells have physiological characteristics that differentiate them from the non-stem cells of developing organs and tumors and have devised indirect means to enrich stem cells from tissue samples or tumors that demonstrably contain stem cells by the ability to reconstitute a tissue, i.e., hematoleukopoietic system or tumor upon introduction into a human or experimental animal. Applicants have previously discovered, however, that these organogenic and carcinogenic stem cells are directly and specifically recognizable on the basis of their distinct nuclear morphology, participation in symmetric and asymmetric amitotic nuclear fission and appendage to cytoplasmic organelles that are rendered brightly fluorescent by Feulgen reagent. These unexpected characteristics were found to be of value in recognizing and enumerating stem cells in tissue biopsies for diagnosis of cancerous or precancerous conditions and in testing the ability of an agent or combination of agents to limit the growth capacity and/or kill pathogenic stem cells. The clear distinction between the eukaryotic cells that constitute the vast majority of cells in a tissue or tumor and these non-mitotic stem cells led to their denomination as a distinct cellular life form: metakaryotic cells.

It is also widely accepted that human genetic lineages are created by copying a double stranded DNA helix (dsDNA/DNA) to form two copies in the form of two sister dsDNA/DNA helices. In formation of the germ cells sister dsDNA/DNA helices are subsequently segregated by the process of meiosis. In parenchymal cells that constitute generally more than 99% of tissue cells of plants and animals sister dsDNA/DNA helices are segregated by the process of mitosis. It has been assumed that the stem cells of organogenesis responsible for growth and development would similarly employ only dsDNA/DNA helices in genome replication and subsequently segregation by mitosis. Applicants have now discovered, however, that the stem cells of human fetal organogenesis as well as the stem cells of human carcinogenesis first create pangenomic copies of the parental dsDNA/DNA genome in the form of two dsRNA/DNA helical copies that are subsequently segregated into two descendant cells by any of several modes of amitosis. During and subsequent to the amitotic segregation process the dsRNA/DNA genomic replicative intermediate is physically associated with enzymes including RNAse-H1, DNA polymerases beta and zeta and other molecules coincident with the transformation of the dsRNA/DNA intermediate into a dsDNA/DNA helix.

Herein are disclosed independent means to recognize and enumerate metakaryotic stem cells undergoing cell division by virtue of the presence of a recognizable mass of dsRNA/DNA in the nuclei of an amitotic fission figure and thus be of value in recognizing and enumerating stem cells in tissue biopsies for diagnosis of cancerous or precancerous conditions, as well as in identifying additional macromolecules and biochemical pathways used by metakaryotes but not eukaryotes during cell division and genome replication that may serve as targets of therapy, and in testing the ability of an agent or combination of agents to limit the growth capacity or kill pathogenic stem cells. It is further disclosed that the dsRNA/DNA helix is itself a specific target for the design and/or selection of therapeutic agents as are the molecules required for formation and segregation of the dsRNA/DNA hybrid genome and for its transformation into a dsDNA/DNA form.

The present invention relates to the prior discovery that metakaryotic stem cells, which in aberrant forms divide and lead to hyperproliferative disorders such as cancer, are characterized by bell-shaped nuclei and undergo a unique form of replication. See Gostjeva, E. V. et al., Cancer Genet. Cytogenet., 164: 16-24 (2006); Gostjeva, E. V. and Thilly, W. G., Stem Cell Rev., 243-252 (2005)). Bell-shaped nuclei divide both symmetrically and asymmetrically by non-mitotic fission processes in colonic and pancreatic human tumors. Gostjeva, E. V., et al., Cancer Genet. Cytogenet., 164:16-24 (2006); Gostjeva, E. V. and Thilly, W. G., Stem Cell Rev., 243-252 (2005). These bell-shaped nuclei appear in great numbers both in 5-7 week embryonic hindgut where they are encased in tubular syncytia, and comprise, for example, 30% of all nuclei and tumor tissues where they abound in “undifferentiated” niches. They possess several stem cell-like qualities, particularly the unique characteristic of asymmetric division and a nuclear fission frequency consistent with growth rates of human colonic preneoplastic and neoplastic tissue (Herrero-Jimenez et al., Mutat. Res. 400:553-78 (1998); Herrero-Jimenez et al., Mutat. Res. 447:73-116 (2000)); see also U.S. Pat. No. 7,427,502 demonstrating, inter alia, that metakaryotes are stem cells. In view of the role of aberrant metakaryotes as cancer stem cells and further in view of the fact that cancer stem cells typically survive standard radio and chemo-therapies, these cells with previously unrecognized nuclear forms are targets for therapeutic strategies.

The present observation that these bell-shaped nuclei undergo a stage where the genome is wholly or substantially represented as an intermediate dsRNA/DNA duplex genome allows for methods of identifying them, diagnostic and prognostic methods, methods of screening therapeutic treatments, as well as methods that target and destroy them by, for example, targeting the DNA polymerases responsible for their replication to double-stranded DNA during amitotic cell division. In certain embodiments, replication complexes comprising DNA polymerase beta and/or DNA polymerase zeta are targeted in the present invention. Replication complexes containing these polymerases can be targeted by, for example, targeting the polymerases directly, either individually or jointly. Thus, the methods provided by the present invention can inhibit DNA polymerase beta activity, DNA polymerase zeta activity, or both DNA polymerase beta activity and DNA polymerase zeta activity, either substantially simultaneously, or sequentially, thus inhibiting the replication of metakaryotic stem cells. In some embodiments, a replication complex comprising RNAse H-1 is targeted, for example, by targeting RNAse H-1 itself. In more particular embodiments RNAse H-1 is targeted in concert with polymerase beta and/or zeta.

### Metakarytotic Cells

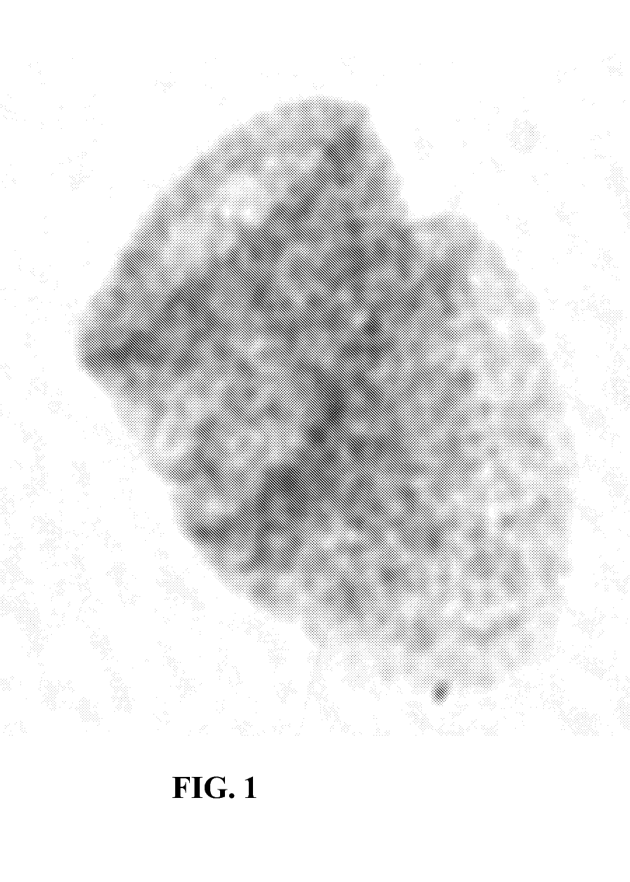

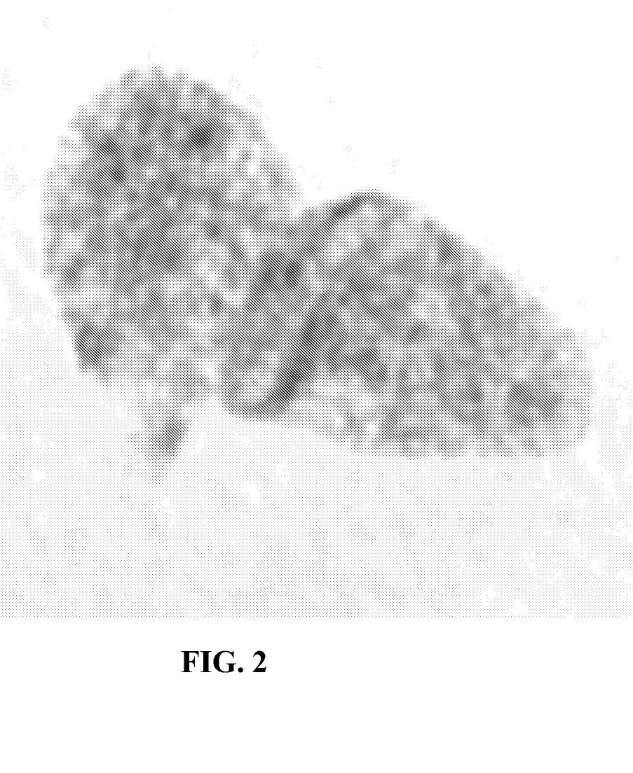

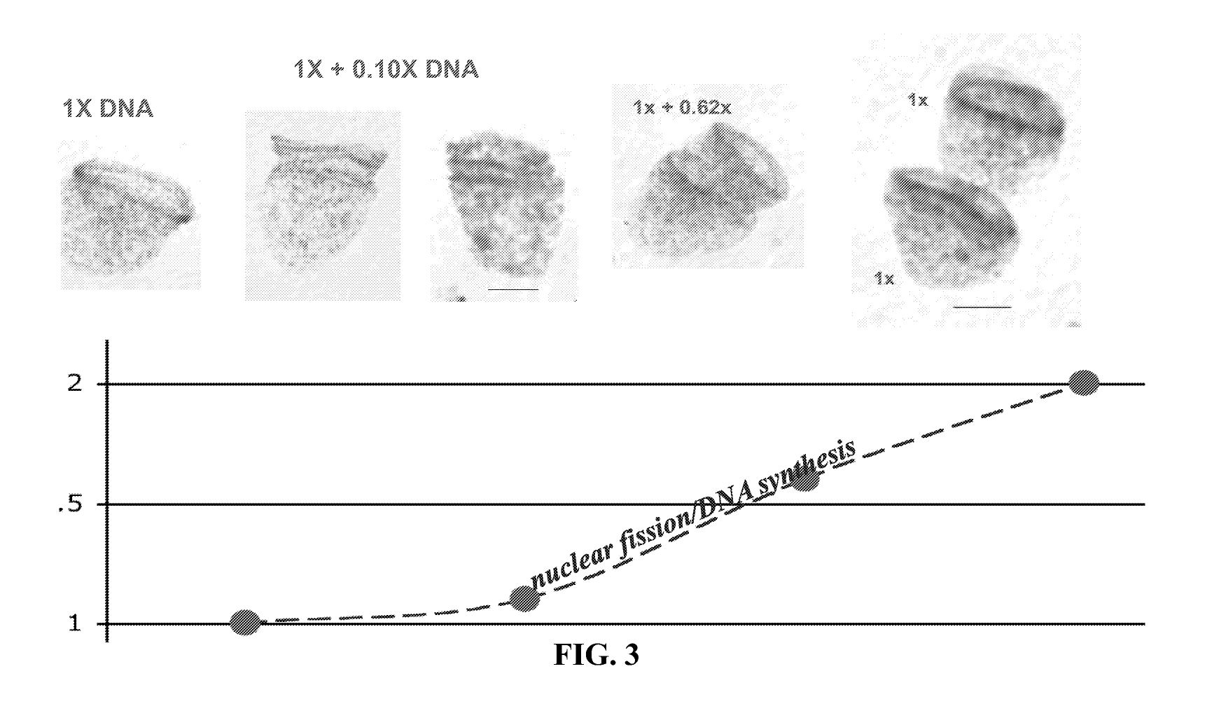

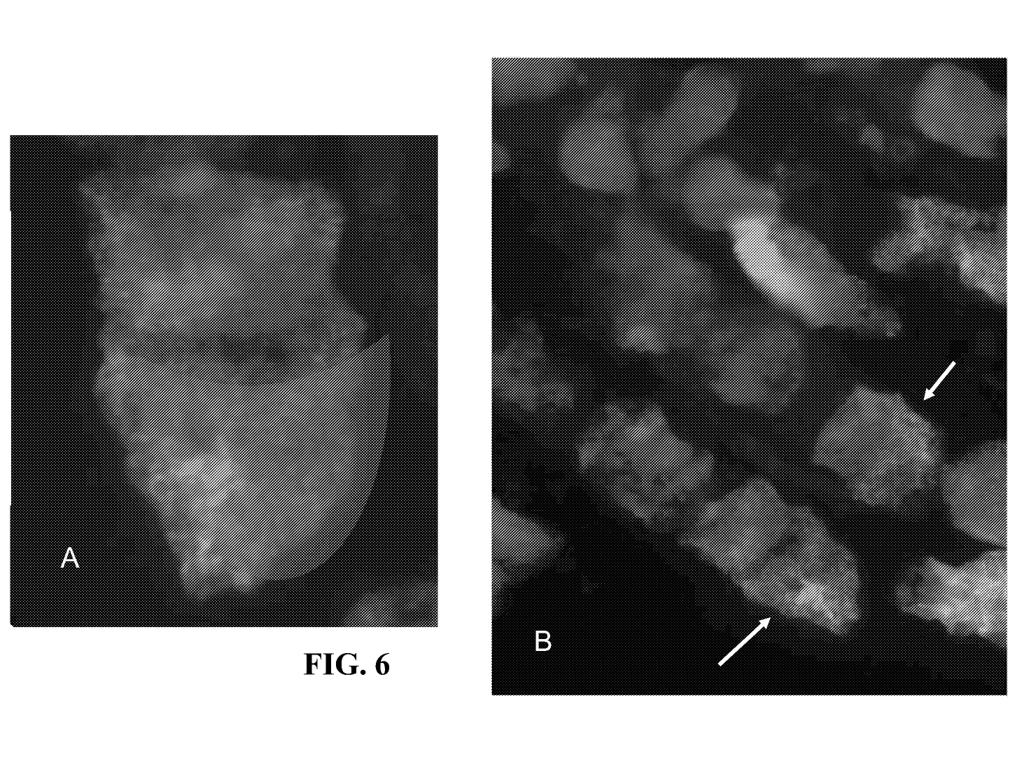

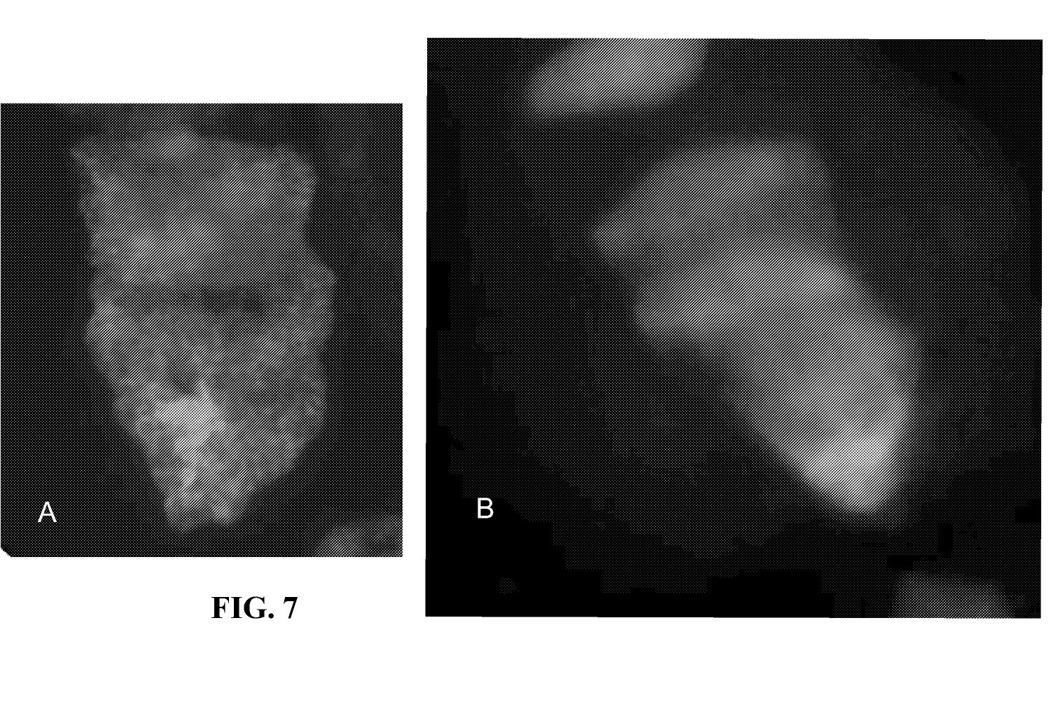

Metakaryotic stem cells exhibit a striking, yet only recently recognized nuclear morphotype: a hollow, bell-shaped nucleus. For a review, see Gostjeva and Thilly, Stem Cell Reviews 2: 243-252 (2005); see also FIGS. 1, 2, 3, 6 and 7 from U.S. Pat. No. 7,427,502 and their descriptions, which are also incorporated by reference in their entirety. These cells also undergo both symmetric (giving rise to additional bell-shaped nuclei) and asymmetric (giving rise to non-bell-shaped nuclei) “amitoses”—division without canonical mitosis and full metaphase chromosome condensation. Through these amitoses, metakaryotic stem cells can give rise to heteromorphic nuclear morphotypes including bell-shaped, cigar-shaped, condensed-spherical, spherical, oval, sausage-shaped, kidney-shaped, bullet-shaped, irregular spindle-shaped, and combinations thereof. See, e.g., FIG. 1 and from U.S. Pat. No. 7,427,502. “Metakaryote,” “metakaryotic stem cell,” “metakaryotic stem cell,” “wound healing metakaryote” and the like, refer to a cell with a hollow, bell-shaped nucleus, where the cell divides by amitosis—either symmetrical or asymmetrical. Metakaryotes have been observed in both animal and plant cells.

The skilled artisan will be able to readily identify metakaryotic stem cells when practicing the methods provided by the invention. For example, the methods of identification, screening, diagnosis, prognosis and treatment provided herein can comprise the step of detecting metakaryotic stem cells from a tissue sample or in cultured cells by detecting an intermediate dsRNA/DNA duplex genome. Cultured cells or cells from within a tissue samples being visualized by the methods of the invention are prepared in a way that substantially preserves the integrity of nuclear structures in nuclei having maximum diameters up to about 10, 20, 30, 40, 50, 60, or 70 microns—and in more particular embodiments up to about 50 microns. Methods for preparing cells are also described in U.S. Pat. No. 7,427,502, the teachings of which are incorporated by reference in their entirety. In certain embodiments, the preparation substantially preserves the integrity of nuclear structures in nuclei of about 10-15 microns. For example, in some embodiments a tissue sample may be analyzed as a preparation of at least about 20, 30, 40, 50, 60, 70, 80, 90, 100, 150, 200, 250, 300, 350, 400, 450, 500, 750, 1000, 1250, 1500 or more microns in thickness. In certain embodiments, a tissue sample is macerated by, for example, incubation in about 45% (e.g., about 25, 30, 35, 40, 42, 45, 47, 50, 55, 60 or 65%) acetic acid in preparation for analysis.

In some embodiments, to further facilitate detection of metakaryotes, cultured cells or tissue samples can be stained. In particular embodiments, the staining can comprise staining with, for example, a Schiff's base reagent, Feulgen reagent, or fuchsin. In more particular embodiments, the tissue sample may be further stained with a second stain. In still more particular embodiments, the second stain may be Giemsa stain.

In certain embodiments, metakaryotic stem cells can be detected by the fluorescence of their cytoplasm, following treatment with a non-fluorescent stain, such as Schiff's reagent. See, e.g., U.S. Patent Application Publication No. 2010/0075366 A1, including Example 5, FIGS. 20-27, and their descriptions, all of which are incorporated by reference. In the present invention “metakaryotic stem cells associated with wound healing disorders,” “wound healing metakaryotes,” and the like, are metakaryotic stem cells that do not exhibit balloon-shaped cytoplasms, in contrast to the large, balloon-shaped cytoplasms of the metakaryotic stem cells described in, e.g., U.S. Patent Application Publication No. 2010/0075366 A1.

Metakaryotic “Cytoplasmic Organelles”

All non-dividing metakaryotic cells discovered to date have hollow concave nuclei (bell shaped nuclei) with a double band of condensed DNA at the rim of the bell mouth. The diameter of the bell mouth is usually some 12-15 microns but the depth of the bell shaped nucleus extends from 3-5 microns in certain tissue types and derived cancers, e.g. in hematopoietic cell preparations from bone marrow or leukemia cells in peripheral circulation to 15-25 microns in some metakaryotes in tumors such as human colonic adenocarcinomas.

In eukaryotes the nucleus is enclosed by a nuclear membrane as an organelle within the volume delimited by the external cellular membrane; usually the nuclei are centrally or near centrally located and the nuclear membrane is not in contact with the cell membrane. In metakaryotic cells, however, there is no obvious nuclear membrane and the hollow nuclei appear to be appended to, rather than enclosed by, the membrane that encloses this cytoplasmic organelle. Certain treatments of human fetal tissue or tumors, e.g., treatment with MATRISPERSE™ for 24 hours at freezing temperature results in physical separation of bell shaped nuclei from cytoplasmic organelles.

Cytoplasmic organelles to which metakaryotic nuclei are eccentrically associated vary in size and dimension. Nearly all are rendered fluorescent by treatment with Feulgen reagent (fuchsin) and are strongly labeled with antibodies for fetal/carcino-mucins. An exception to the strong labeling for mucins in cytoplasmic organelles are the metakaryotic cells giving rise to the smooth muscle cells in vascularization of fetal organs and the pathological condition of post-surgical restenosis.

Cytoplasmic organelles may be nearly spherical bodies associated with a shallow bell shaped nucleus. These are the smallest metakaryotic cells, less than 15 micron in diameter, observed by Applicants. Applicants teach that these smallest metakaryotic cells with near spherical cytoplasmic organelles and nuclei appended to them as shallow bell shaped nuclei resembling yarmulkes (skullcaps) are those described in the literature as “signet ring” cells often noted in development of some organs, e.g. gastric pits, hematopoiesis and certain hematopoietic diseases, e.g., leukemias. Applicants teach that these smallest metakaryotes constitute an important stem cell lineage in tissues or disease status such as leukemias where they are found.

Cytoplasmic organelles may also be prolate spheroids or balloon shaped with very great lengths. Examples of metakaryotic cytoplasmic organelles greater than 200 microns have been observed by Applicants in human tumors.

In addition, in some embodiments, metakaryotes can be detected and/or further characterized by detecting particular marker genes that have proven useful in indirect methods for enriching a bone marrow, solid tissue or tumor sample for stem cells that are inferred to be present by transplant and xenotransplant assays of the “enriched” cell material. In particular embodiments, the marker genes can include one or more of CD133 (prominin 1; human GeneID 8842, reference mRNA and protein for the longest isoform are NM_{—}006017.2 and NP_{—}006008.1, respectively) and CD44 (human GeneID 960, reference mRNA and protein sequences for the isoform 1 precursor are NM_{—}000610.3 and NP_{—}000601.3, respectively). The marker genes may be detected at the nucleic acid (e.g., RNA) or protein level. In more particular embodiments, the marker genes may be detected at the periphery of a balloon-shaped cytoplasm of a metakaryote. The foregoing GeneIDs may be used to retrieve publicly-available annotated mRNA or protein sequences from the NCBI website. The information associated with these GeneIDs, including reference sequences and their associated annotations, are all incorporated by reference. Reference sequences from other organisms may readily be obtained from the NCBI website as well. However, Applicants also teach that markers such as CD133 and CD44 are found throughout tissues and tumors associated with non-metakaryotic cells and other non-cellular structures.

The invention is based, in part, on the discovery that metakaryotic stem cells can be specifically identified by detecting an intermediate of an amitosis, which is unexpectedly associated with an intermediate dsRNA/DNA duplex genome. “Intermediate dsRNA/DNA duplex genome” or “intermediate dsRNA/DNA hybrid genome” or “dsRNA/DNA hybrid genome” and the like are a replication intermediate of metakaryotic stem cells where a substantial fraction of the previously dsDNA/DNA nuclear genome is in the form of double-stranded nucleic acid comprising a strand of ribonucleic acid hybridized to a complementary strand of deoxyribonucleic acid—a dsRNA/DNA duplex. A substantial fraction refers to at least 0.2, 0.3, 0.4, 0.5, 1, 2, 3, 4, 5, 10, 20, 30, 40, 42, 45, 47, 50, 52, 55, 57, 60, 62, 65, 67, 70, 80, 90, 95, 99%, or more, of the nuclear DNA being in the dsRNA/DNA duplex form. In particular embodiments, a substantial fraction refers to at least 50, 90, 95, 99% or more of the nuclear DNA being in the dsRNA/DNA duplex form. In other embodiments, a substantial fraction is about 1-24 picograms, e.g., about 0.5, 1, 2, 3, 4, 5, 6, 7, 8, 9, 10, 12, 14, 16, 18, 20, 22, or 24 pg. This unique structure stands in stark contrast to, e.g., viral genomes, which, inter alia, may utilize extremely small dsRNA/DNA hybrid genome. In addition, the intermediate dsRNA/DNA duplex genome of metakaryotic stem cells is readily differentiated from reports of dsRNA/DNA hybrids in eukaryotic cells during, e.g., transcription, since such reports are not of replication intermediates. Furthermore, it has been estimated that only relatively minor fractions of the genome are in a dsRNA/DNA duplex—0.01-0.1%. See, e.g., Szeszak and Pihl Biochem. Biophys. Acta 247: 363-67 (1971), Alcover et al., Chromosoma 8: 263-77 (1982). Accordingly, as may be used in this application, a “metakaryotic amitosis” is the amitotic division (either symmetrical or asymmetrical) of a metakaryote, and is associated with an intermediate dsRNA/DNA duplex genome.

Detecting an Intermediate dsRNA/DNA Duplex Genome

The replicative intermediate dsRNA/DNA duplex genome of metakaryotic stem cells can be detected by a variety of means, such as by detecting proteins involved in the amitosis (discussed below) and/or by detecting the dsRNA/DNA duplex itself.

A dsRNA/DNA duplex can be detected by using nucleic acid dyes that discriminate between single-stranded and double-stranded nucleic acids. Dyes that “discriminate between single-stranded and double-stranded nucleic acids” exhibit a differential affinity for single-stranded and double-stranded nucleic acids and/or exhibit different spectral properties (e.g., excitation, adsorption, or emission spectra) when bound to single-stranded vs. double-stranded nucleic acids. Certain dyes may be colorimetric, e.g., fluorescent, while others may not be colorimetric but have affinity for particular nucleic acids, and may therefore be conjugated to additional molecules for visualization.

For example, in some embodiments, dyes that discriminate between single-stranded and double-stranded nucleic acids exhibit greater affinity for double stranded nucleic acids and include dyes such as DAPI or Hoechst dyes, such as Hoechst 33342 or Hoechst 33258. For example, DAPI does not stain dsRNA/DNA but does stain dsDNA/DNA. Where a metakaryotic nucleus is in the 100% dsDNA/DNA form it will appear blue, while a nucleus in a ˜100% dsRNA/DNA form will not exhibit detectable DAPI staining. In other embodiments, dyes that discriminate between single-stranded and double-stranded nucleic acids exhibit greater affinity for single-stranded nucleic acids and include dyes such as TOTO®3 and OLIGREEN® (INVITROGEN®). Other dyes that discriminate between single-stranded and double-stranded nucleic acids exhibit different spectral properties when bound to single-stranded or double-stranded nucleic acids, and include acridine orange, which fluoresces red when bound to single-stranded nucleic acids, and green when bound to double-stranded nucleic acids. In some embodiments, dyes that discriminate between single-stranded and double-stranded nucleic acids can also have enhanced affinity for dsRNA/DNA hybrids and include the molecules described in Table 3 of Shaw and Arya Biochimie 90:1026-39 (2008), which is incorporated by reference in its entirety, and includes ethidium bromide, propidium iodide, ellipticine, actinomycin D and derivatives (such as N8 or F8 AMD), paramomycin, ribostamycin, neomycin, and the neomycin-methidium chloride conjugate “NM,” as well as lexitropsins and polyamides, including distamycin (such as bis-distamycins, particularly ortho/para) and netropsin.

Dyes that discriminate between single-stranded and double-stranded nucleic acids can be used alone or in conjunction with conjugates to facilitate visualization (such as in the case of dyes that are not themselves colorimetric) and may further be used in concert with RNAse. For example, RNAse may be useful in the identification of a dsRNA/DNA duplex through detecting a change in the expected colorimetric reaction of a particular dye discussed above. A particular example is the spectral shift observed when acridine orange binds single-stranded nucleic acids instead of double-stranded nucleic acids. Accordingly, in certain embodiments, a dsRNA/DNA duplex is detected by acridine orange staining after RNAse treatment, which leaves only the single-stranded DNA of the duplex. Other analogous approaches can be adapted for use in the methods provided by the invention. For example, Table 1, below, provides agents that bind single-stranded DNA and can therefore be used in the methods provided by the invention, following degradation of RNA (e.g., by alkali or, preferably, RNAse treatment).

TABLE 1
Chemical agents that bind single-stranded DNA.

| Chemical Agent | Mechanism |
|---|---|
| Actinomycin D | Extensively studied anti-tumor agent. |
| | Binds to hairpin DNA structures abundant |
| | in single-stranded DNA. Blocks RNA |
| | synthesis from DNA template. Possible |
| | dsRNA/DNA binding agent. |
| Bromoacetaldehyde | Reacts at the base-pairing positions of |
| | adenines and cytosines. Preferentially |
| | reacts with bases in single-stranded loops |
| | and cruciforms. |
| Chloroacetaldehyde | A metabolite of vinyl chloride that readily |
| | interacts with single-stranded DNA to |
| | predominantly form etheno lesions. |
| Diethyl | Carboxyethylates purines at the N-7 |
| pyrocarbonate | position, which opens the imidazole ring. |
| | Substantially reactivity toward single- |
| | stranded regions of DNA. |
| Osmium tetroxide | Adds to the C-5, C-6 double bond of |
| | pyrimidines in the presence of pyridine to |
| | form osmate esters. Substantially more |
| | reactive to single-stranded DNA than |
| | double-stranded DNA. |
| Potassium | Pyrimidine-specific and single-strand |
| permanganate | specific. Modifies bases via oxidation. |

In certain embodiments a dsRNA/DNA duplex is detected using antibodies. The term “antibody,” as used herein, refers to an immunoglobulin or an antigen-binding fragment thereof, and encompasses any polypeptide comprising an antigen-binding site regardless of the source, species of origin, method of production, or characteristics. As a non-limiting example, the term “antibody” includes human, orangutan, rabbit, mouse, rat, goat, sheep, and chicken antibodies. The term includes but is not limited to polyclonal, monoclonal, monospecific, polyspecific, non-specific, humanized, camelized, single-chain, chimeric, synthetic, recombinant, hybrid, mutated, and CDR-grafted antibodies. For the purposes of the present invention, it also includes, unless otherwise stated, antibody fragments such as Fab, F(ab′)2, Fv, scFv, Fd, dAb, VHH (also referred to as nanobodies), and other antibody fragments that retain antigen-binding function. Antibodies also include antigen-binding molecules that are not based on immunoglobulins, as further described below.

Antibodies can be made, for example, via traditional hybridoma techniques (Kohler and Milstein, Nature 256: 495-499 (1975)), recombinant DNA methods (U.S. Pat. No. 4,816,567), or phage display techniques using antibody libraries (Clackson et al., Nature 352: 624-628 (1991); Marks et al., J. Mol. Biol, 222: 581-597 (1991)). For various other antibody production techniques, see Antibodies: A Laboratory Manual, eds. Harlow et al., Cold Spring Harbor Laboratory, 1988.

In some embodiments, the term “antibody” includes an antigen-binding molecule based on a scaffold other than an immunoglobulin. For example, non-immunoglobulin scaffolds known in the art include small modular immunopharmaceuticals (see, e.g., U.S. Patent Application Publication Nos. 2008/0181892 and 2008/0227958 published Jul. 31, 2008 and Sep. 18, 2008, respectively), tetranectins, fibronectin domains (e.g., AdNectins, see U.S. Patent Application Publication No. 2007/0082365, published Apr. 12, 2007), protein A, lipocalins (see, e.g., U.S. Pat. No. 7,118,915), ankyrin repeats, and thioredoxin. Molecules based on non-immunoglobulin scaffolds are generally produced by in vitro selection of libraries by phage display (see, e.g., Hoogenboom, Method Mol. Biol. 178:1-37 (2002)), ribosome display (see, e.g., Hanes et al., FEBS Lett. 450:105-110 (1999) and He and Taussig, J. Immunol. Methods 297:73-82 (2005)), or other techniques known in the art (see also Binz et al., Nat. Biotech. 23:1257-68 (2005); Rothe et al., FASEB J. 20:1599-1610 (2006); and U.S. Pat. Nos. 7,270,950; 6,518,018; and 6,281,344) to identify high-affinity binding sequences.

Immunogens to generate antibodies specific for dsRNA/DNA duplexes useful in the methods provided by the invention include, for example, poly(A)/poly(dT) (see, e.g., Kitagawa and Stollar Mol. Immunol. 19: 413-20 (1982) and U.S. Pat. No. 4,732,847 at 6:2-14, which are incorporated by reference), poly(dC)/poly(I) (see, e.g., Kitagawa and Stollar 1982), and φX174 dsRNA/DNA hybrid (see, e.g, Nakazato Biochemistry 19:2835-40 (1980) or U.S. Pat. No. 5,200,313 at 14:53-15:13, which are incorporated by reference). Accordingly, in certain embodiments, an antibody for use in the methods of the invention binds at least one antigen selected from poly(A)/poly(dT), poly(dC)/poly(I), and φX174 dsRNA/DNA hybrid; or another double-stranded nucleic acid molecule comprising one strand of RNA and one strand of DNA with complementary mixed base sequences. In particular embodiments, the antibody binds one or more of these antigens with a Ka of greater than about 1×10⁶, 5×10⁶, 1×10⁷, 5×10⁷, 1×10⁸, 5×10⁸, 1×10⁹ M⁻¹, or more. Specific antibodies for use in the methods provided by the invention include the antibody produced by the hybridomas deposited with the ATCC® (American Type Culture Collection) under accession numbers ATCC HB 8730, HB 8076, HB 8077, and HB 8078, as well as chimeria and CDR-grafted variants of these antibodies.

In other embodiments, antibodies specific for single-stranded DNA may be used in the methods provided by the invention after degradation of the RNA in the duplex, such as following RNAse treatment. Suitable immunogens for generating ssDNA-binding antibodies include denatured preparations of DNA, such as calf thymus DNA. ssDNA-specific antibodies can be induced by immunization of animals with complexes of methylated BSA complexes and ssDNA (Plescia et al., PNAS, 52: 279, 1964) or synthetic ss polynucleotides (Seaman et al., Biochemistry, 4: 2091, 1965) or with fragments of DNA conjugated to proteins (Table 1 of Stollar, Nucleic Acid Antigens, in The Antigens Vol 1, M. Sela Ed., Academic Press, 1973). SSDNA-specific antibodies can also be obtained as polyclonal autoantibodies from sera of some patients with systemic lupus erythematosus (Stollar and Levine, J. Immunol., 87: 477, 1961, Arch. Biochem Biophys. 101:417, 1963), or lupus mice (Munns and Freeman, Biochemistry, 28: 10048, 1989)); or as monoclonal autoantibodies from human or mouse hybridomas (Shoenfeld et al., J. Clin. Invest., 70: 205, 1982; Andrzejewsky et al., J. Immunol, 126, 226, 1981; Eilat, D Molec Immunol. 31:1377, 1994).

In particular embodiments, the antibody for ssDNA is a monoclonal antibody, such as the monoclonal antibody Mab F7-26 (MILLIPORE® cat no. MAB3299), or a chimeric or CDR-grafted variant thereof. Other agents with ssDNA binding specificity may also be used to detect ssDNA after removal of RNA from dsRNA/DNA hybrids, including single-stranded oligonucleotides (including ssDNA, RNA, PNA or other artificial nucleic acids capable of hybridizing to ssDNA), or proteins with ssDNA specificity, including, for example, poly(ADP-ribose) polymerase, hnRNP proteins, single-stranded DNA binding protein and RecA.

Any of the agents for use in the methods provided by the invention, such as dsRNA/DNA duplex or ssDNA antibodies may be detectably labeled. Alternatively, the agents may not be labeled and may be detected indirectly using a secondary agent, e.g., a detectably labeled secondary antibody. Detectable labels may be enzymatic (e.g., HRP or alkaline phosphatase), fluorescent, radiolabels, chemical moieties (small molecules, such as biotin), protein moieties (such as avidin or polypeptide tags), et cetera.

### Proteins Involved in Amitoses

By the present invention, Applicants have identified several of the molecules likely to be involved in the amitotic replication of metakaryotes, including DNA polymerase beta, DNA polymerase zeta, and RNAseH1. Accordingly, in the methods provided by the invention, an intermediate dsRNA/DNA duplex genome can be identified by detecting the dsRNA/DNA duplex itself, e.g., by the methods described above, or by detecting the expression products (at the nucleic acid or protein level) of genes involved in replication of metakaryotic stem cells, such as polymerases beta and zeta, RNAseH1, and combinations thereof, including combinations in concert with detecting the dsRNA/DNA duplex.

DNA polymerase beta is one of the major DNA repair polymerases in the base-excision repair (BER) pathways. DNA polymerase beta is a 39 kDa protein and the major BER polymerase (GenBank accession number NM 002690), but in contrast to the high fidelity replicative DNA polymerases, DNA polymerase beta lacks 3′ to 5′ exonuclease activity and proof-reading Capabilities, resulting in reduced fidelity. Chyan, Y., et al., Nucleic Acids Res. vol. 22, no. 14, pp. 2719-2725 (1994). Polymerase beta genes have been identified in a number of organisms, such as those identified in Table 2.

TABLE 2
PolBeta genes

| Species | GeneID |
|---|---|
| Homo sapiens | 5423 |
| Mus musculus | 18970 |
| Rattus norvegicus | 29240 |
| Bos taurus | 614688 |
| Gallus gallus | 426794 |
| Pan troglodytes | 737210 |
| Canis lupus familiaris | 494001 |

An example of an error-prone DNA polymerase is DNA polymerase zeta, a 173 kDa protein encoded by the Rev3 gene (Gibbs, P. E. M., et al., Proc. Natl. Acad. Sci. USA, vol. 95, pp. 6876-6880 (1998); GenBank Accession number AF058701)). DNA polymerase zeta is a translesion synthesis polymerase which bypasses DNA damage by incorporating a nucleotide opposite a sequence lesion rather than repairing it, allowing synthesis to continue with the mismatched nucleotide remaining in the sequence (Gan, G. N., et al., Cell Res. 18: 174-183 (2008)). Polymerase zeta genes have been identified in a number of organisms, such as those in Table 3.

TABLE 3
Pol Zeta/REV3L genes

| Species | GeneID |
|---|---|
| Homo sapiens | 5980 |
| Saccharomyces cerevisiae | 855936 |
| Pan troglodytes | 462942 |
| Rattus norvegicus | 309812 |
| Mus musculus | 19714 |
| Macaca mulatta | 695894 |
| Ailuropoda melanoleuca | 100466621 |
| Canis lupus familiaris | 481963 |
| Xenopus laevis | 100316923 |

RNAse H1 cleaves the RNA strand of dsRNA/DNA duplexes. Assays for RNAseH1 activity are known in the art and are described in, for example, paragraph 32 of U.S. Patent Application Publication No. 20050014708 A1, which is incorporated by reference. RNAseH genes have been identified in a variety of organisms, such as those reported in Table 4. In addition, MMDB ID: 63294 provides a structure of the hybrid-binding domain of human RNAse H1 in complex with 12-mer RNA/DNA. This structure can be used, for example, in the rational design and selection of therapeutics for use in the methods provided by the invention.

TABLE 4
RNAse H1 genes

| Species | GeneID |
|---|---|
| Homo sapiens | 246243 |
| Mus musculus | 19819 |
| Rattus norvegicus | 298933 |
| Equus caballus | 100072832 |
| Pan troglodytes | 743012 |
| Bos taurus | 613354 |
| Gallus gallus | 395848 |
| Danio rerio | 436932 |

These gene identifiers in Tables 2-4 may be used to retrieve, inter alia publicly-available annotated mRNA or protein sequences from sources such as the NCBI website, //www.ncbi.nlm.nih.gov. The information associated with these identifiers, including reference sequences and their associated annotations, are all incorporated by reference. Additional useful tools for converting IDs or obtaining additional information on a gene are known in the art and include, for example, DAVID, Clone/GeneID converter, and SNAD. See Huang et al., Nature Protoc. 4(1):44-57 (2009), Huang et al., Nucleic Acids Res. 37(1)1-13 (2009), Alibes et al., BMC Bioinformatics 8:9 (2007), Sidorov et al., BMC Bioinformatics 10:251 (2009).

Additional macromolecules, such as proteins (as well as lipids, carbohydrates, and nucleic acids), involved in metakaryotic amitosis can be identified by methods provided by the invention, e.g., by detecting a candidate macromolecule by colocalization with an intermediate dsRNA/DNA duplex genome. Macromolecules (and their associated biochemical pathways) can then be targeted as described in, for example, the next section.

### Inhibitors of Proteins Involved in Amitoses

Polymerases beta and zeta, as well as RNAseH1 can be inhibited by routine means in the art, such as neutralizing antibodies, dominant negative mutants, and nucleic-acid based techniques, such as antisense, siRNA, and triplex forming oligonucleotides. Other inhibitors are known in the art.

Inhibitors of polymerase beta include, for example, those disclosed in paragraphs 49, 50, and Table 1 of U.S. Patent Application Publication No. 2010/0048682, which are incorporated by reference. Additional polymerase beta inhibitors include those described in Wilson et al. Cell Mol Life Sci. 67(21):3633-47 (2010) and Yamaguchi et al., Biosci Biotechnol Biochem. 74(4):793-801 (2010; describing novel terpenoids and trichoderonic acids A and B).

RNAse H1 inhibitors include triplex forming oligonucleotides (see WO 94/05268, Duval-Valentin et al., Proc. Natl. Acad. Sci. USA 89: 504-508 (1992); Fox, Curr. Med. Chem., 7:17-37 (2000); Praseuth et al., Biochim. Biophys. Acta, 1489: 181-206 (2000)). Other inhibitors include 1-hydroxy-1,8-naphthyridine compounds, such as those disclosed in paragraphs 43-101 of U.S. Patent Application Publication No. 2010/0056516 A1 and the compounds disclosed in the summary of invention in U.S. Pat. No. 7,501,503, which are incorporated by reference. Other RNAseH1 inhibitors can include agents that target the dsRNA/DNA duplex, such as aminoglycosides including neomycin, kanamycin, paromomycin, tobramycin and ribostamycin.

Additional RNAseH1 inhibitors include those referenced in the background section of U.S. Patent Application Publication No. 2010/0056516 A1, including substituted thienes (see, e.g., WO2006/026619 A2), dithiocarbamates (see, e.g., U.S. Patent Application Publication No. 2005/0203176 A1), dihydroquinoline derivatives (see, e.g., U.S. Patent Application Publication No. 2005/0203129 A1), hydantoin derivatives (see, e.g., U.S. Patent Application Publication No. 2005/0203156 A1), oligonucleotide agents (see, e.g., US 2004/0138166 A1), mappicine related compounds (see, e.g., U.S. Pat. No. 5,527,819), thiophene derivatives (see, e.g., WO 2006/026619 A2), carbamate derivatives (see, e.g., U.S. Patent Application Publication No. 2005/203176 A1), hydantoins (see, e.g., U.S. Patent Application Publication No. 2005/203156 A1), 1,2-dihydroquinoline derivatives (see, e.g., U.S. Patent Application Publication No. 2005/203129 A1), lactones (see, e.g., Dat, et al., Journal of Natural Products, 70: 839-841 (2007)), hydroxylated tropolones (see, e.g., Didierjean, et al., Antimicrobial Agents and Chemotherapy, 49: 4884-4894 (2005)), hydroxylated tropolones (see, e.g., Budihas et al., Nucleic Acids Res. 33: 1249-56 (2005)), DNA thioaptamers (see, e.g., Somasunderam et al., Biochemistry 44: 10388-95 (2005)), diketoacid (see, e.g., Shaw-Reid et al., Biochemistry 44: 1595-1606 (2005) and Shaw-Reid et al., J. Biol. Chem. 278: 2777-80 (2003)), oligonucleotide hairpins (see, e.g., Hannoush et al., Nucleic Acids Res. 32: 6164-6175 (2004)), 2-hydroxyisoquinoline-1,3(2H,4H)-dione (see, e.g., Klumpp et al., Nucleic Acids Res. 31: 6852-59 (2003) and Qi Hang et al., Biochem. Biophy. Res. Comm. 317: 321-29 (2004)), acylhydrazone (see, e.g., G. Borko et al., Biochemistry, 36: 3179-3185 (1997)), novenamines (see, e.g., Althaus et al., Experimentia 52 Birkhauser-Verlag, pp. 329-335) (1996)), naphthalenesulfonic acid derivatives (see, e.g., Mohan et al., J. Med. Chem., 37: 2513-2519 (1994)), cephalosporin degradation product (see, e.g., P. Hafkemer et al., Nucleic Acids Res. 19: 4059-65 (1991)), and quinone (see, e.g., Loya et al., Antimicrobial Agents and Chemother. 34: 2009-12 (1990)).

The compounds above, including combinations thereof, such as, at least 1, 2, 3, 4, 5, or more of the compounds above, can be used in the methods provided by the invention to inhibit a replication complex associated with an intermediate dsRNA/DNA duplex genome comprising one or more of DNA polymerase beta or zeta, and/or RNAseH1.

### Methods

The invention provides diagnostic, prognostic and treatment methods for a variety of disorders in any organism comprising metakaryotic cells. Exemplary methods include the diagnosis, prognosis, and/or treatment of tumors, non-cancerous hyperproliferative disorders and wound healing disorders, as well as methods of identifying metakaryotic stem cells, screening for agents that modulate the growth, migration, replication, and/or survival of metakaryotic stem cells and can therefore be used in the treatment methods provided by the invention and to identify additional targets for anti-stem cell therapy by discovering macromolecules or biochemical pathways present or expressed in amitotic metakaryotic stem cells containing a dsRNA/DNA hybrid genome. The methods of the present invention include the step of identifying metakaryotic stem cell undergoing amitosis associated with an intermediate dsRNA/DNA hybrid genome—i.e., a metakaryotic amitosis. Any metakaryotic cell, e.g., animal or a multicellular plant (Gostjeva et al., 2009) can be used in methods of identifying metakaryotic cells, as well as in methods of screening for agents or discovering macromolecules or biochemical pathways.

### Subjects and Tissue Samples

Subjects to be diagnosed, prognosed, screened, or treated by the methods provided by the invention include any organism comprising metakaryotic cells. In certain embodiments, the organism is a multicellular animal, such as a vertebrate. In particular embodiments, the subject may be a mammal, such as a primate, a rodent, a canine, a feline, a porcine, an ovine, a bovine, or a leporine. In still more particular embodiments, the subject is a primate, e.g., a human. In other embodiments, the subject is a rodent.

In other embodiments, the subject is a plant, e.g., the invention provides methods for identify pathological disease states and mechanisms in plants and, in other aspects, provides methods for identifying agents that modulate the growth, migration, and/or proliferation of metakaryotic stem cells in plants, such as, for example, herbicides.

The invention provides diagnostic, prognostic and treatment methods for tumors, non-cancerous hyperproliferative disorders and wound healing disorders, as well as methods of screening for agents that modulate the growth, migration, replication, and/or survival of metakaryotic stem cells and can therefore be used in the treatment methods provided by the invention. The methods of the present invention include the step of identifying metakaryotic stem cell undergoing amitosis associated with an intermediate dsRNA/DNA hybrid genome—i.e., a metakaryotic amitosis.

The subject may be at any stage of development, e.g., an embryo, a fetus, a neonate, infant, child, adolescent, adult, or geriatric. In particular embodiments, the subject is a child, adolescent, adult, or geriatric. In still more particular embodiments, the subject is an adult or geriatric. In certain embodiments, the subject is at least about 1, 2, 3, 4, 5, 10, 20, 25, 30, 35, 40, 45, 50, 55, 60, 65, 70, 75, or more years old, e.g., about 1-5, 5-10, 10-20, 18-25, 25-35, 35-45, 45-55, 55-65, or 65-75 years old, or greater. In certain embodiments, the subject is deceased, i.e., the method is a post-mortem diagnostic method.

In certain embodiments, a tissue sample from a subject is obtained surgically, e.g., during a surgery such as a transplant, angioplasty, or stenting, or in a biopsy procedure. The tissue sample may include tumor tissue, non-tumor tissue, or a combination thereof and may include tissues such as, blood, vascular tissue, adipose tissue, lymph tissue, connective tissue (e.g., fascia, ligaments, tendons), adventitia, serosa, aponeuroses, endocrine tissue, mucosal tissue, liver, lung, kidney, spleen, stomach, pancreas, colon, small intestine, bladder, gonad, mammary tissue, central nervous tissue, peripheral nervous tissue, skin, smooth muscle, cardiac muscle, or skeletal muscle. In some embodiments a tissue sample may comprise 1, 2, 3, 4, 5, or more of the above tissues. In more particular embodiments a tissue sample may comprise or consist essentially of primarily one tissue, e.g., the tissue sample is about 10, 20, 30, 40, 50, 60, 70, 80, 90, 95, or 100% by weight of a single tissue. In more particular embodiments, the tissue sample comprises blood vessel tissue and in still more particular embodiments, blood vessel wall tissue. In some embodiments, the issue sample consists essentially of blood vessel tissue. In certain embodiments, the blood vessel tissue further comprises adventitia. In more particular embodiments, the blood vessel tissue consists essentially of adventitia and blood vessel tissue. In certain particular embodiments the tissue sample comprises suspected tumor tissue.

### Diagnostic Methods

The diagnostic methods provided by the invention comprise determining (e.g., measuring) the presence and/or quantity and/or migration of metakaryotic stem cells in a tissue sample from a subject to diagnose a disorder in the subject, such as a tumor, a wound healing disorder, or a non-cancerous hyperproliferative disorder. In a particular embodiment, the presence and/or quantity and/or distribution of metakaryotic stem cells undergoing metakaryotic amitosis is determined.

### Disorders for Diagnosis

A variety of disorders can be diagnosed using the methods provided by the invention including tumors, wound healing disorders, and non-cancerous hyperproliferative disorders.

A “wound healing disorder” is a disease or disorder characterized by aberrant tissue generation during the repair of damage to tissues and/or organs following surgical intervention, recovery from infection (such as a flesh-eating infection), and/or acute trauma, where the aberrant tissue generation is non-cancerous and non-precancerous. Somatic cells of “non-cancerous” and “non-precancerous” growth(s) exhibit normal (wild-type) chromosomal karyotypes and normal contact inhibition when cultured. In some embodiments, the wound healing disorder is characterized by aberrant excessive tissue generation. In other embodiments, the wound healing disorder is characterized by aberrant inadequate tissue generation. Exemplary wound healing disorders include blood vessel wound healing disorders, spinal cord wound healing disorders, wound healing disorders associated with organ transplants and wounds associated with traumatic injuries. In more particular embodiments, the wound healing disorder is post-surgical. Surgery, such as organ transplant e.g., heart, liver, lung, cornea, et cetera or surgical intervention, such as angioplasty, stent placement, et cetera, often leads to restenosis (arterial or veinous). Such restenosis is the frequent cause of death in transplant recipients. Acute traumas can include, for example, burns, cuts and gunshot wounds.

A “blood vessel wound healing disorder” is a wound healing disorder in vascular tissue. In certain embodiments a blood vessel wound healing disorder is characterized by aberrant excessive smooth muscle generation and/or proliferation of metakaryotic cells in vascular tissue, particularly luminal surfaces, such as the intima. Exemplary blood vessel wound healing disorders include, for example, injury-induced neointimal hyperplasia and restenosis (e.g., following transplantation or trauma). In more particular embodiments, the blood vessel wall disorder is restenosis. “Restenosis” refers to a re-narrowing of an artery, typically by a thickening of the intimal surface, following surgical intervention such as angioplasty, stenting, or transplantation. In some embodiments, the blood vessel wall disorder occurs after surgery, infection, or acute trauma. In more particular embodiments, the blood vessel wall disorder is post-surgical.

Accordingly, in some embodiments of diagnostic or prognostic methods provided by the invention, the subject is suspected of having a wound healing disorder. In more particular embodiments, the subject is suspected of having a blood vessel wound healing disorder.

In certain embodiments, a subject suspected of having a wound healing disorder has previously undergone surgery. In more particular embodiments, the surgery is a stenting and/or balloon angioplasty. In still more particular embodiments, the subject has previously received more than one stent, e.g., at least 2, 3, 4, 5, or more stents. In these embodiments the stents may be drug-eluting (e.g., sirolimus or paclitaxel-eluting, including analogs thereof; as well as anti-CD-34 or anti-VEGF antibody-coated stents), non-drug-eluting, or combinations thereof.

In some embodiments, a subject suspected of having a wound healing disorder has previously received a transplant, e.g., an allograft, autograft, or xenograft. In particular embodiments the subject has had a complete or partial organ transplant (e.g., heart, liver, kidney, bladder, skin, lung, or cornea transplant), or a valve or vessel transplant. The transplanted vessels may be either arteries and/or veins. In particular embodiments, the subject is suspected of having restenosis following surgery.

The skilled artisan will appreciate that de novo disorders, such as atherosclerosis, are not wound healing disorders for the purposes of the present invention. Nevertheless, the invention provides, in certain embodiments, methods for diagnosing non-cancerous hyperproliferative disorder, such as atherosclerosis, by visualizing tissues suspected of containing a non-cancerous hyperproliferative lesion by the methods of the invention. For example, the nuclei of cells in a sample, such as a biopsy, are visualized and the presence of bell-shaped nuclei undergoing amitosis associated with an intermediate dsRNA/DNA duplex genome is determined.

A “tumor” refers to a neoplastic growth and encompasses both benign and malignant neoplasms as recognized by surgical pathologists on the basis of rate of growth, position degree of healthy tissue invasion, metastasis and the presence of actively dividing mitotic cells and cells with irregularly shaped and stained, dysplastic nuclei. Practicing the present invention, a pathologist would be able to observe and enumerate the nuclei of specimen strongly reacting with anti dsRNA/DNA antibody or dye that specifically stains dsRNA/DNA molecules. In a typical adenocarcinma, such as of the colon, the metakaryotic stems cells would be expected to undergo symmetric or asymmetric mitoses about every twelve days and constitute about 0.2 to 2% of the metakaryotic cells in a tumor specimen. From Herrero-Juminez et al. 1988, 2000.

“Preneoplastic lesions” refer to small, slow growing squamous or adenomatous bodies associated with tumors as presumptive precursors as is the case for adenomatous polyps of the colon with potentially metastatic adenocarcinomas of that tissue. As symmetric divisions of preneoplastic stem cells are expected but once in 5-6 years, the frequency of asymmetric divisions of about once every forty days would lead to the expectation that only about 1/4000 metakaryotic nuclei would be found with a dsRNA/DNA hybrid genome in a preneoplastic lesion such as an adenomatous colonic polyp.

In some embodiments the disorder is monoclonal; i.e., the disorder arises by linear growth from a single metakaryotic stem cell vis-à-vis asymmetrical divisions to form an aberrant excessive tissue growth. In other embodiments, the disorder is polyclonal, i.e., the disorder arises from two or more metakaryotic cells by both symmetrical and asymmetrical divisions, e.g., post-surgical restenosis.

### Screening Methods

The invention provides both in vitro and in vivo methods of screening for agents to modulate the growth, replication, migration, and/or survival of metakaryotic stem cells. In both the in vitro and in vivo methods, candidate agents are evaluated for their ability to modulate the number and/or migration of cells containing bell-shaped nuclei undergoing amitosis associated with an intermediate dsRNA/DNA duplex genome. A candidate agent can comprise any chemical entity, including a small molecule pharmaceutical or biologic, such as a protein (e.g., growth factor, antibody, or aptamer), nucleic acid (including antisense molecules and aptamers), lipid, carbohydrate, or combinations thereof. The agent will typically be administered at dose or a range of doses, e.g., 2, 3, 4, 5, 6, or more doses, so as to elicit an effect on the number of bell-shaped nuclei undergoing amitosis associated with an intermediate dsRNA/DNA duplex genome, in the culture or organism.

In vitro screening methods comprise contacting cultured cells comprising proliferating metakaryotic stem cells with a candidate agent. In more particular embodiments, the cells are obtained from an animal, such as a vertebrate, such as a mammal, such as a primate, a rodent, a canine, a feline, a porcine, an ovine, a bovine, or a leporine. In still more particular embodiments, the cells are obtained from a human. In certain embodiments, the cultured cells are obtained from umbilical cord, adventitia, mesenchymal tissue, or aortic arch. In other embodiments, the cultured cells are obtained from a tumor, such as a solid tumor, such as breast, prostate, lung, or colon tumor. In particular embodiments, the cultured cells are HT29 human colon adenocarcinoma cells, as described in Example 6 of U.S. Patent Application Publication No. 2010/0075366 A1, including FIGS. 28-30, and their descriptions, all of which are incorporated by reference. In still other embodiments, the metakaryotic cells are from a plant. In these embodiments, for example, it is possible to screen for herbicides that target metakaryotic cells specific to an undesirable plant (e.g. a weed), but not a desirable plant (e.g., a crop).

In certain particular embodiments, the cultured cells comprise proliferating metakaryotic stem cells and muscle cells. In still more particular embodiments, the cells are primary cells. In more particular embodiments, the primary cells are obtained from umbilical cord, vascular adventitia, or aortic arch.

Cultures can be enriched for metakaryotic stem cells in a variety of ways. In certain embodiments, the culture is treated with ionizing radiation, such as X-rays, at a dose sufficient to kill most eukaryotic cells, but not metakaryotic stem cells, owing to their exceptional radiation-resistance. In particular embodiments, the cells are x-irradiated at a dose of 150, 200, 250, 300, 350, 400, 500, 600, 700, 800, 1000, 1600 rads, or more. In more particular embodiments, the cells are x-irradiated at a dose of greater than 400 rads, such as 800 rads or 1600 rads.

In vivo screening methods comprise administering a candidate agent to an organism comprising metakaryotic stem cells, e.g., an animal or plant. In particular embodiments, the organism is an animal, and in still more particular embodiments, a mammal. In yet more particular embodiments, the mammal is a non-human mammal. In still more particular embodiments, the mammal is a non-human primate, a rodent, a canine, a feline, a porcine, an ovine, a bovine, or a leporine. In yet still more particular embodiments the mammal is a rodent, such as a mouse, rat or guinea pig. In still more particular embodiments, the mammal is a guinea pig. In some embodiments, the mammal is predisposed (e.g., genetically or via diet or drug treatment) to develop a wound healing disorder, non-cancerous hyperproliferative disorder, or tumor. For example, in certain embodiments, a wound healing disorder arises from a surgical intervention, e.g., surgical insult such as transplantation, angioplasty, stenting, or direct intentional tissue damage, e.g., by chemical fixation, radiation, excess heat or cold, infarct, stabbing, cutting, or blunt trauma. In more particular embodiments, the mammal is both predisposed to develop a wound healing disorder and is exposed to a surgical intervention. In certain embodiments the wound healing disorder is a blood vessel wound healing disorder. In more particular embodiments, the blood vessel wound healing disorder is restenosis.

In other embodiments, the organism is predisposed to develop a tumor or has a tumor. In more particular embodiments, the organism is an animal, such as a mammal predisposed to develop a tumor or has a tumor. In certain embodiments, the mammal may be a transgenic animal engineered to express an oncogene (e.g., RAS or HER2) or is a knockout or hypomorph for a tumor suppressor gene (e.g., p53), or a combination thereof and/or may be given a mutagenic treatment. In other embodiments, the organism is a xenotransplant, for example, is transplanted with human tumor cells. In more particular embodiments, the tumor cells are from a solid tumor. In particular embodiments, the mammal is a rodent, such as a rat, mouse, or guinea pig. The xenograft is allowed to mature into solid tumor, which could then be excised and further studied. In these embodiments, the rodent will typically be immune-compromised. One of ordinary skill in the art is familiar with xenotransplant techniques.

### Treatment Methods

The screening methods described above provide agents that can be used to treat disorders driven by aberrant metakaryotic stem cell activity, such as wound healing disorders, non-cancerous hyperproliferative disorders, or tumors. Therefore, the invention also provides methods of treating a subject having a disorder driven by aberrant metakaryotic stem cell activity. For example, a subject with any wound healing disorder can be administered an effective amount of an agent that modulates, e.g., the number of proliferating metakaryotic stem cells, or the migration of proliferating metakaryotic stem cells. For example, in wound healing disorders characterized by aberrant excessive tissue generation, the subject is administered an agent that decreases the number of proliferating metakaryotic stem cells, or the migration of proliferating metakaryotic stem cells. Conversely, in wound healing disorders characterized by aberrant inadequate tissue generation, the subject is administered an effective amount of an agent that increases the number of proliferating metakaryotic stem cells, or the migration of proliferating metakaryotic stem cells. “Agent” refers to both single-active agent compounds as well as combinations of active agents.

In a subject with a tumor, the subject is administered an agent that modulates the number, migration, replication, or survival of metakaryotic stem cells. In particular embodiments, the agent reduces the number, migration, replication, or survival of metakaryotic stem cells. In more specific embodiments, the agent reduces the number of replicating metakaryotic cells undergoing amitosis associated with an intermediate dsRNA/DNA hybrid genome. In other more specific embodiments, the agent transiently increases the number of replicating metakaryotic cells undergoing amitosis associated with an intermediate dsRNA/DNA hybrid genome, but inhibits completion of replication.

The term, “treatment” refers to ameliorating symptoms associated with the disorder, including, for example, reducing, preventing or delaying metastasis of a carcinoma; reducing the number, volume, and/or size of one or more tumors; and/or to lessening the severity, duration or frequency of symptoms of the carcinoma or pathology as well as modulating the number of metakaryotic stem cells, the number of proliferating (by symmetrical or asymmetrical amitosis) metakaryotic stem cells, and/or the migration of metakaryotic stem cells.

Generally, agents/conditions that inhibit eukaryotic dsDNA/DNA synthesis and mitosis are expected to constitute a generally non-overlapping set of agents/conditions that inhibit metakaryotic dsRNA/DNA to dsDNA/DNA processing and the processes of symmetric and/or asymmetric amitoses. This is because chemicals found to shrink tumors in clinical practice, but which are followed by tumor re-emergence are not expected to be useful in killing metakaryotic stem cells or ultimately curing cancer. This is because said metakaryoic cells are strongly resistant to killing by x-irradiation and treatment with radio-mimetic drugs such as alkylating agents and agents that attack mitosis or eukaryotic modes of DNA replication not employed by metakaryotes.

Nevertheless, in some embodiments, the methods of treatment for a subject with a tumor provided by the invention are used in conjunction with one or more of surgery, hormone ablation therapy, radiotherapy or chemotherapy. The chemotherapeutic, hormonal and/or radiotherapeutic agent and treatment according to the invention may be administered simultaneously, separately or sequentially. For example, in some embodiments, a subject may be treated with one or more “metakaryocides” (an agent that kills or reduces the number of metakaryotes) to eliminate tumor stem cells, as well as a therapy to eliminate the non-stem cell tumor mass.

In certain embodiments, a therapeutic agent for use in the methods provided by the invention comprises an active agent component/moiety and a targeting agent component/moiety. The targeting agent component is or comprises an agent that specifically binds to dsRNA/DNA duplexes, as described herein. In particular embodiments, the targeting agent comprises any of the agents described above, such as antibodies, or antigen binding fragments thereof. The targeting agent component is linked to the active agent component. For example, they can be covalently bonded directly to one another or through a linker molecule. Where the two are directly bonded to one another by a covalent bond, the bond may be formed by forming a suitable covalent linkage through an active group on each moiety. For instance, an acid group on one compound may be condensed with an amine, an acid or an alcohol on the other to form the corresponding amide, anhydride or ester, respectively. In addition to carboxylic acid groups, amine groups, and hydroxyl groups, other suitable active groups for forming linkages between a targeting agent component and an active agent component include sulfonyl groups, sulfhydryl groups, and the haloic acid and acid anhydride derivatives of carboxylic acids.

In another embodiment, the therapeutic agent can comprise two, or more, moieties or components, typically a targeting agent moiety with one or more active agent moieties. Linkers can be used to link an active agent to a targeting agent component, wherein the targeting agent specifically interacts with a dsRNA/DNA hybrid thereby delivering the active agent to the replicative intermediate configuration, and inhibiting further replication of the bell-shaped nuclei.

The active agent component, which is linked to the targeting agent component, can be or comprise any agent that achieves the desired therapeutic result, including agents such as: a radionuclide (e.g., I125, 123, 124, 131 or other radioactive agent); a chemotherapeutic agent (e.g., an antibiotic, antiviral or antifungal); an immune stimulatory agent (e.g., a cytokine); an anti-neoplastic agent: an anti-inflammatory agent; a pro-apoptotic agent (e.g., peptides); a toxin (e.g., ricin, enterotoxin, LPS); an antibiotic; a hormone; a protein (e.g., a surfactant protein, a clotting protein, as well as growth factors); a lytic agent; a small molecule (e.g., inorganic small molecules, organic small molecules, derivatives of small molecules, composite small molecules); nanoparticles (e.g., lipid or non-lipid based formulations); lipids; lipoproteins; lipopeptides; liposomes; lipid derivatives; a natural ligand; an altered protein (e.g., albumin or other blood carrier protein-based delivery system); a nucleolytic enzyme; an agent that modulates growth or migration of the tumor stem cell; a gene or nucleic acid (e.g., an antisense oligonucleotide); viral or non-viral gene delivery vectors or systems; or a prodrug or promolecule. One skilled in the art will be familiar with the design and application of the active agent.

With the selective targeting of dsRNA/DNA hybrids in a limited population of vulnerable tumor cells, it is reasonable to believe that a dosing regimen can be recalculated for improved efficacy.

The following examples are provided to illustrate the resent invention and are not intended to be limiting in any way.

### Exemplification:

The following general protocols were used to generate the data shown in the figures and described in the specification. Experimental conditions are summarized in Table 5.

TABLE 5

| Pro- | 0.1% Triton-X- | Blocking | Blocking | Primary | Secondary | |
|---|---|---|---|---|---|---|
| tocol | 100 | solution n1 | solution n2 | antibodies | antibodies | DAPI |
| N1 | 1 h 30′ | 1% BSA + 3 | None | 1:10, over | Donkey anti-goat | 1:1000 |
| | | drops of | | night in | IgG-TRITC, 1:200, | |
| | | normal goat | | refrigerator | 1 h, room | |
| | | serum | | | temperature | |
| N2 | 40′ for syncytia; | 1% BSA, 1 h | 5% normal | 1:10, over | Bovine anti-goat | 1:1000 |
| | 20′ for cells | | bovine | night in | IgG-TRITC, 1:200, | |
| | | | serum, 1 h | refrigerator | 1 h, room | |
| | | | | | temperature | |
| N3 | 40′ | 1% BSA, 1 h | 5%, normal | 1:10, over | Donkey anti-goat | 1:1000 |
| | | | goat serum, | night in | IgG-TRITC, 1:200, | |
| | | | 1 h | refrigerator | 1 h, room | |
| | | | | | temperature | |
| N4 | 40′ for syncytia; | 1% BSA, 1 h | 5%, normal | 1:10, over | Donkey anti-goat | 1:1000 |
| | 20′ for cells | | goat serum, | night in | IgG-TRITC, 1:200, | |
| | | | 1 h | refrigerator | 1 h, room | |
| | | | | | temperature | |
| N5 | 40′ for syncytia; | 1% BSA | 5%, normal | 1:20, over | Donkey anti-goat | 1:1000 |
| | 20′ for cells | | goat serum, | night in | IgG-TRITC, 1:200, | |
| | | | 1 h | refrigerator | 1 h, room | |
| | | | | | temperature | |
| N6 | 40′ for syncytia; | 1% BSA | 5%, normal | 1:20, over | Donkey anti-goat | 1:1000 |
| | 20′ for cells | | goat serum, | night in | IgG-TRITC, 1:200, | |
| | | | 1 h | refrigerator | 1 h, room | |
| | | | | | temperature | |
| N7 | 1 h20′ | 1% BSA | 5% normal | 1:20, over | Donkey anti-goat | 1:1000 |
| | | | donkey | night in | IgG-TRITC, 1:200, | |
| | | | serum, 1 h | refrigerator | 1 h, room | |
| | | | | | temperature | |
| N8 | 20′ | 1% BSA | 5% normal | 1:20, over | Donkey anti-goat | 1:1000 |
| | | | donkey | night in | IgG-TRITC, 1:200, | |
| | | | serum, 1 h | refrigerator | 1 h, room | |
| | | | | | temperature | |

Due to syncytial autofluorescence causing high background noise and thick syncytial cell walls, which prevent antibodies from crossing the walls, staining syncytia required incubation with Triton-X-100 to permeabilize the cell membranes. In addition, it was necessary to add a second blocking agent to decrease the background noise (blocking solution n2 in Table 5).

Experiments to detect a dsRNA-DNA duplex were carried out using the following antibodies: 1) Goat 4A-E, IgG absorbed with poly(dT), lot JH012680, 1.07 mg/ml stock solution, 0.05 and 0.1 mg/ml final assay concentration at 1:20 and 1:10 dilutions, respectively; 2) Goat 4H, IgG absorbed with poly(dT), poly(A), and poly(A).poly(U) to remove any reactivity with denatured DNA or dsRNA, A280=3.4, 2.4 mg/ml stock solution, 0.12 mg/ml final assay concentration at 1:20 dilution; and 3) Goat 4A-E, IgG absorbed with poly(dT), lot 021580, A280=3.2, 2.3 mg/ml stock solution, 0.12 mg/ml final assay concentration at 1:20 dilution.

Each of the three foregoing antibodies were tested individually on the following tissues: 1) Human fetal tissue, 9-10 weeks, spinal cord or intercostals muscle prep; 2) Human fetal colon; 3) Human colon adenocarcinoma, M.68; 4) HT-29 cell line, DMEM (Dulbecco's Modified Eagle's Medium), 5% horse serum or DMEM, 10% BSA; and 5) HT-29 cell line, DMEM, 5% horse serum, irradiated 1600 RAD.

The following protocol was used for fixation and IF (immunofluorescence) staining for dsRNA/DNA. All tissues, including HT-29 cells, fetal and neoplastic tissues were fixed with Carnoy fixative for 3 hours. Carnoy's solution was 3:1, ethanol (4° C.): glacial acetic acid (mixed together just before fixation). Fixative was replaced three times with fresh samples in the duration of three hours. Carnoy's fixative was replaced with 70% methanol and stored at 4° C. Slides were prepared by spreading fetal or neoplastic tissue (following 1-hour incubation of the tissue with collagenese II (Calbiochem, 100 mg (activity 277 U/mg), diluted to 15 U/ml working concentration), 37° C., followed by spreading in a drop of 45% acetic acid to achieve milder conditions of maceration). This spreading/maceration step was omitted in experiments with HT-29 cells.

Slides with the tissue were then air dried. Slides were next transferred into 1×PBS buffer for 5 minutes and were then treated with 0.1% Triton X-100 in 1×PBS at room temperature for 20-80 minutes (see Table 5). Next, slides were washed twice with 1×PBS wash buffer for 5 minutes. 1% BSA in 1×PBS (blocking solution n1) was then applied for 60 minutes at room temperature. 5% Donkey serum in 1×PBS (blocking solution n2) was then applied for 60 minutes at room temperature.

Primary dsRNA/DNA-specific antibody was diluted to a working concentration (1:20-1:10, resulting in working concentrations of 0.05 mg/ml (sometimes 0.1 mg/ml at 1:10 dilution), 0.12 mg/ml, and 0.12 mg/ml for preparations 1, 2, and 3, respectively) in 0.1% BSA in 1×PBS solution, and was incubated overnight in a refrigerator (4° C.).

Next, slides were washed three times (10 minutes each) with 1×PBS buffer. Secondary antibody (TRITC-conjugated, Santa-Cruz) diluted in 1×PBS was prepared just prior to use (1:200) and incubated with the slide for 60 minutes at room temperature. Slides were washed three times (10 minutes each) with 1×PBS buffer.

Nuclei counterstaining was then performed by incubating the tissue with DAPI (MILLIPORE® Corp., 0.1 mg/ml stock solution, diluted 1:1000) for 1-5 minutes at room temperature, followed by washing tissue three times (5-10 minutes each) with 1×PBS buffer.

The specificity of staining by antibodies was checked for by the blocking test with poly(A)-poly(dT), which was performed as follows:

### Test Poly(A)-Poly(dT) as Blocker at 10 Ug/Ml.

Equal volumes of Poly(A)-Poly(dT) (20 ug/ml) and antibody (1/10) were mixed to give final concentrations of 10 ug/ml A.dT and 1/20 of serum (antibody). For controls, PBS was used in place of Poly(A)-Poly(dT). Samples were incubated for 10-15 minutes at room temperature.

### Test Varying Concentrations of Poly(A)-Poly(dT)

Samples were then tested at final concentrations of 10, 2.0, 0.5 and 0.08 micrograms/ml, in each case incubating equal volumes of polynucleotide and antibody that are twice the final concentration, as above. The staining with dsRNA/DNA-specific antibodies was negative and completely blocked by the addition of 10 micrograms/milliliter of Poly(A)-Poly(dT).

It should be understood that for all numerical bounds describing some parameter in this application, such as “about,” “at least,” “less than,” and “more than,” the description also necessarily encompasses any range bounded by the recited values. Accordingly, for example, the description at least 1, 2, 3, 4, or 5 also describes, inter alia, the ranges 1-2, 1-3, 1-4, 1-5, 2-3, 2-4, 2-5, 3-4, 3-5, and 4-5, et cetera.

For all patents, applications, or other reference cited herein, such as non-patent literature and reference sequence information, it should be understood that it is incorporated by reference in its entirety for all purposes as well as for the proposition that is recited. Where any conflict exits between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures) are hereby incorporated by reference in their entirety.

Headings used in this application are for convenience only and do not affect the interpretation of this application.

While this invention has been particularly shown and described with references to preferred embodiments thereof, it will be understood by those skilled in the art that various changes in form and details may be made therein without departing from the scope of the invention encompassed by the appended claims.

1. A method for identifying a metakaryotic stem cell, comprising the steps of:
a) visualizing the nuclei of cells in a sample, wherein the sample is prepared by a method that substantially preserves the integrity of nuclei; and
b) identifying cells containing an intermediate dsRNA/DNA duplex genome in the sample,
thereby identifying a metakaryotic stem cell.

2. The method of claim 1, wherein the metakaryotic stem cell is an animal stem cell.

3. The method of claim 1, further comprising c) enumerating the cells identified in step b).

4. The method of claim 1, wherein the metakaryotic stem cell is selected from a fetal, juvenile, adult, or tumor stem cell.

5. The method of claim 2, wherein the animal is a mammal, such as a human.

6. The method of claim 3, wherein the sample is an isolated tissue biopsy sample or a cell culture sample.

7. The method of claim 1, wherein the visualization is a result of contacting the cells with: a detectably labeled antibody specific for a dsRNA/DNA duplex, or a detectably labeled antibody specific for ssDNA and the sample being contacted with RNAse before visualization of the nuclei with the antibody specific for ssDNA.

8. The method of claim 7, wherein the label is fluorescent.

9. The method of claim 1, wherein the visualization is a result of contacting the cells with a dye that discriminates between single-stranded and double-stranded nucleic acids.

10-13. (canceled)

14. The method of claim 6, wherein the sample is a tissue biopsy from tissue suspected of containing a tumor, wound healing lesion or atherosclerotic lesion, wherein the wound healing lesion may optionally be a restenotic lesion.

15. A method for identifying an agent that modulates the growth, migration, replication, or survival of a metakaryotic stem cell, comprising:
a) determining the presence and/or number and/or distribution of nuclei comprising an intermediate dsRNA/DNA duplex genome in cells in a sample, wherein the sample comprises metakaryotic stem cells comprising an intermediate dsRNA/DNA duplex genome and was maintained in contact with a candidate agent under conditions suitable for the agent to interact with the nuclei, and wherein the sample was prepared by a method that substantially preserves the integrity of nuclei; and
b) comparing the presence and/or number of nuclei comprising an intermediate dsRNA/DNA duplex genome in the cells contacted with the candidate agent to the presence and/or number of nuclei comprising an intermediate dsRNA/DNA duplex genome in control cells comprising metakaryotic stem cells but not contacted with the candidate agent,
whereby a change in the number and/or distribution of nuclei comprising an intermediate dsRNA/DNA duplex genome in the cells contacted with the candidate agent, relative to the control cells not contacted with the candidate agent, is indicative of the effectiveness of the agent.

16. The method of claim 15, wherein the cells are contacted with the candidate agent in culture.

17. The method of claim 16, wherein the cells are animal cells.

18. The method of claim 15, wherein the animal cells are contacted with the candidate agent in vivo.

19. (canceled)

20. The method of claim 15, wherein the visualization is a result of: contacting the cells with a detectably labeled antibody specific for a dsRNA/DNA duplex, or contacting the cells with a detectably labeled antibody specific for ssDNA and the sample being contacted with RNAse before visualization of the nuclei with the antibody specific for ssDNA.

21. The method of claim 20, wherein the antibody is fluorescently labeled.

22. The method of claim 15, wherein the visualization is a result of contacting the cells with a dye that discriminates between single-stranded and double-stranded nucleic acids.

23-29. (canceled)

30. The method of claim 15, wherein a decrease in the number of bell-shaped nuclei undergoing metakaryotic amitosis in the cells contacted with the candidate agent is detected.

31. A method for identifying macromolecules associated with metakaryotic stem cell duplication comprising detecting a candidate macromolecule in a metakaryotic stem cell containing an intermediate dsRNA/DNA duplex genome, wherein co-localization of the candidate macromolecule with the intermediate dsRNA/DNA duplex genome indicates that the macromolecule is associated with metakaryotic stem cell duplication.

32. (canceled)

33. The method of claim 31, wherein the metakaryotic stem cell is an animal stem cell.

34. (canceled)
